SEQUENCE LISTING (59 sequences)

SEQ ID NO: 1 - protein, 10 aa, Homo sapiens
  VARIANT 5: note = X is Gly or Pro
CPTGXQNYSP

SEQ ID NO: 2 - protein, 10 aa, Homo sapiens
PGIPGRNGDP

SEQ ID NO: 3 - protein, 15 aa, Homo sapiens
PDGSESPTDQETTGV

SEQ ID NO: 4 - protein, 10 aa, Homo sapiens
CPTGPQNYSP

SEQ ID NO: 5 - protein, 11 aa, Homo sapiens
  VARIANT 5: note = X is Gly or Pro
CPTGXQNYSPQ

SEQ ID NO: 6 - protein, 9 aa, Homo sapiens
  VARIANT 5: note = X is Pro or Gly
CPTGXQNYS

SEQ ID NO: 7 - protein, 5 aa, Mus musculus
NYVIH

SEQ ID NO: 8 - protein, 17 aa, Mus musculus
YMNPYNDVPKNNAKFRG

SEQ ID NO: 9 - protein, 9 aa, Mus musculus
GGFFGPLSY

SEQ ID NO: 10 - protein, 16 aa, Mus musculus
RSSQNIVYSNGDTYFE

SEQ ID NO: 11 - protein, 7 aa, Mus musculus
KVSQRFS

SEQ ID NO: 12 - protein, 9 aa, Mus musculus
FQGAHDPPA

SEQ ID NO: 13 - protein, 79 aa, Mus musculus
RSSQNIVYSNGDTYFEWYLQKPGQSPKLLIYKVSQRFSGVPDRFSGSGSGTDFTLKISRVETEDLGVYYCFQGAHDPPA

SEQ ID NO: 14 - protein, 77 aa, Mus musculus
NYVIHWLKQKAGQGPEWIGYMNPYNDVPKNNAKFRGKARLTSDRSSTTAYMELNSLTSEDSAVYYCARGGFFGPLSY

SEQ ID NO: 15 - protein, 112 aa, Mus musculus
DVLMTQTPLSLSVSLGDQASISCRSSQNIVYSNGDTYFEWYLQKPGQSPKLLIYKVSQRFSGVPDRFSGSGSGTDFTLKISRVETEDLGVYYCFQGAHDPPAFGGGTKLELK

SEQ ID NO: 16 - protein, 118 aa, Mus musculus
EVQLQQSGPEVLKPGASVKMSCKASGYTFINYVIHWLKQKAGQGPEWIGYMNPYNDVPKNNAKFRGKARLTSDRSSTTAYMELNSLTSEDSAVYYCARGGFFGPLSYWGQGTLVTVSA

SEQ ID NO: 17 - protein, 10 aa, Homo sapiens
KPGVISVMGT

SEQ ID NO: 18 - protein, 11 aa, Homo sapiens
KPGVISVMGTA

SEQ ID NO: 19 - protein, 9 aa, Homo sapiens
KPGVISVMG

SEQ ID NO: 20 - protein, 16 aa, Mus musculus
RSSQRIVHSNGITFLE

SEQ ID NO: 21 - protein, 7 aa, Mus musculus
RVSNRFS

SEQ ID NO: 22 - protein, 9 aa, Mus musculus
FQGSHVPLT

SEQ ID NO: 23 - protein, 5 aa, Mus musculus
DFNMN

SEQ ID NO: 24 - protein, 17 aa, Mus musculus
AINPHNGATSYNQKFSG

SEQ ID NO: 25 - protein, 7 aa, Mus musculus
WGNGKNS

SEQ ID NO: 26 - protein, 79 aa, Mus musculus
RSSQRIVHSNGITFLEWYLQKPGQSPKLLIYRVSNRFSGVPDRFSGSGSGTDFTLKISRVEAEDLGLYYCFQGSHVPLT

SEQ ID NO: 27 - protein, 75 aa, Mus musculus
DFNMNWVKQSHGKSLEWIGAINPHNGATSYNQKFSGKATLTVDKSSSTAYMELNSLTSDDSAVYYCARWGNGKNS

SEQ ID NO: 28 - protein, 112 aa, Mus musculus
DVVMTQTPLSLPVNLGDQASISCRSSQRIVHSNGITFLEWYLQKPGQSPKLLIYRVSNRFSGVPDRFSGSGSGTDFTLKISRVEAEDLGLYYCFQGSHVPLTFGAGTRLELK

SEQ ID NO: 29 - protein, 116 aa, Mus musculus
EVQLQQSGPVMVKPGTSVKTSCKASGYTFTDFNMNWVKQSHGKSLEWIGAINPHNGATSYNQKFSGKATLTVDKSSSTAYMELNSLTSDDSAVYYCARWGNGKNSWGQGTTLTVSS

SEQ ID NO: 30 - protein, 16 aa, Homo sapiens
PDGSESPTDQETTGVE

SEQ ID NO: 31 - protein, 14 aa, Homo sapiens
DGSESPTDQETTGV

SEQ ID NO: 32 - protein, 11 aa, Homo sapiens
PPGIPGRNGDP

SEQ ID NO: 33 - protein, 11 aa, Homo sapiens
PGIPGRNGDPG

SEQ ID NO: 34 - protein, 9 aa, Homo sapiens
GIPGRNGDP

SEQ ID NO: 35 - protein, 9 aa, Homo sapiens
PGIPGRNGD

SEQ ID NO: 36 - protein, 55 aa, Homo sapiens
GPNVCALQQILGTKKKYFSTCKNWYKKSICGQKTTVLYECCPGYMRMEGMKGCPA

SEQ ID NO: 37 - protein, 55 aa, Homo sapiens
GPNVCAVQKVIGTNRKYFTNCKQWYQRKICGKSTVISYECCPGYEKVPGEKGCPA

SEQ ID NO: 38 - protein, 8 aa, Homo sapiens
  VARIANT 7: note = X is V or L
GPNVCAXQ

SEQ ID NO: 39 - protein, 7 aa, Homo sapiens
YECCPGY

SEQ ID NO: 40 - protein, 7 aa, Homo sapiens
  VARIANT 2: note = X is E or M
GXKGCPA

SEQ ID NO: 41 - protein, 10 aa, synthetic construct
GSPGKDGVRG

SEQ ID NO: 42 - protein, 448 aa, Mus musculus
EVQLQQSGPEVLKPGASVKMSCKASGYTFINYVIHWLKQKAGQGPEWIGYMNPYNDVPKNNAKFRGKARLTSDRSSTTAYMELNSLTSEDSAVYYCARGGFFGPLSYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 43 - protein, 219 aa, Mus musculus
DVLMTQTPLSLSVSLGDQASISCRSSQNIVYSNGDTYFEWYLQKPGQSPKLLIYKVSQRFSGVPDRFSGSGSGTDFTLKISRVETEDLGVYYCFQGAHDPPAFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 44 - protein, 440 aa, Mus musculus
EVQLQQSGPVMVKPGTSVKTSCKASGYTFTDFNMNWVKQSHGKSLEWIGAINPHNGATSYNQKFSGKATLTVDKSSSTAYMELNSLTSDDSAVYYCARWGNGKNSWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 45 - protein, 219 aa, Mus musculus
DVVMTQTPLSLPVNLGDQASISCRSSQRIVHSNGITFLEWYLQKPGQSPKLLIYRVSNRFSGVPDRFSGSGSGTDFTLKISRVEAEDLGLYYCFQGSHVPLTFGAGTRLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 46 - protein, 71 aa, Homo sapiens
GYQLCSHRVTHTVQKVEAVQTSYTSYVSCGGWIPWRRCPKMVYRTQYLVVEVPESRNVTDCCEGYEQLGLY

SEQ ID NO: 47 - protein, 76 aa, Homo sapiens
DPNTCSFWESFTTTTKESHSRPFSLLPSEPCERPWEGPHTCPQPTVVYRTVYRQVVKTDHRQRLQCCHGFYESRGF

SEQ ID NO: 48 - protein, 75 aa, Homo sapiens
DPNVCSHWESYAVTVQESYAHPFDQIYYTRCTDILNWFKCTRHRISYKTAYRRGLRTMYRRRSQCCPGYYESGDF

SEQ ID NO: 49 - protein, 75 aa, Homo sapiens
DPNVCSHWESYSVTVQESYPHPFDQIYYTSCTDILNWFKCTRHRVSYRTAYRHGEKTMYRRKSQCCPGFYESGEM

SEQ ID NO: 50 - protein, 77 aa, Homo sapiens
MPHVCAEQELTLVGRRQPCVQALSHTVPVWKAGCGWQAWCVGHERRTVYYMGYRQVYTTEARTVLRCCRGWMQQPDE

SEQ ID NO: 51 - protein, 78 aa, Homo sapiens
GRNWCPYPMSKLVTLLALCKTEKFLIHSQQPCPQGAPDCQKVKVMYRMAHKPVYQVKQKVLTSLAWRCCPGYTGPNCE

SEQ ID NO: 52 - protein, 75 aa, Homo sapiens
GKNWCAYVHTRLSPTVILDNQVTYVPGGKGPCGWTGGSCPQRSQKISNPVYRMQHKIVTSLDWRCCPGYSGPKCQ

SEQ ID NO: 53 - protein, 76 aa, Homo sapiens
NKNWCAYIVNKNVSCSVLEGSESFIQAQYNCAWNQMPCPSALVYRVNFRPRYVTRYKTVTQLEWRCCPGFRGGDCQ

SEQ ID NO: 54 - protein, 75 aa, Homo sapiens
HRNWCAYVVTRTVSCVLEDGVETYVKYQPCAWGQPQCPQSIMYRRFLRPRYRVAYKTVTDMEWRCCQGYGGDDCA

SEQ ID NO: 55 - protein, 76 aa, Homo sapiens
HKALCAYVVHRNVTCILQEGAESYVKAEYRQCRWGPKCPGTVTYRTVLRPKYKVGYKTVTDLAWRCCPGFTGKRCP

SEQ ID NO: 56 - protein, 76 aa, Homo sapiens
RRHWCHHTVTRTVSCQVQNGSETVVQRVYQSCRWPGPCANLVSYRTLIRPTYRVSYRTVTVLEWRCCPGFTGSNCD

SEQ ID NO: 57 - protein, 73 aa, Homo sapiens
RRNWCSYVVTRTISCHVQNGTYLQRVLQNCPWPMSCPGSSYRTVVRPTYKVMYKIVTAREWRCCPGHSGVSCE

SEQ ID NO: 58 - protein, 73 aa, Homo sapiens
SQGVCSKQTLVVPLHYNESYSQPVYKPYLTLCAGRRICSTYRTMYRVMWREVRREVQQTHAVCCQGWKKRHPG

SEQ ID NO: 59 - protein, 71 aa, Homo sp.
GRRVCAVRAHGDPVSESFVQRVYQPFLTTCDGHRACSTYRTIYRTAYRRSPGLAPARPRYACCPGWKRTSG

(19) United States
(12) Patent Application Publication
(10) Pub. No.: US 2026/0258118 A1
(43) Pub. Date: Sep. 3, 2026

(54) Fibroblast Activation Markers and TGFBI in Therapy

(71) Applicant: KeyBioscience SA, Lugano (CH)

(72) Inventors: Nicholas Willumsen, Gentofte (DK); Cecilie Bager, København V (DK); Neel Nissen, Slangerup (DK); Kim Henriksen, Hillerod (DK); Morten Asser Karsdal, København Ø (DK)

(73) Assignee: KeyBioscience SA, Lugano (CH)

(21) Appl. No.: 19/159,996

(22) PCT Filed: Feb. 27, 2024
(86) PCT No.: PCT/EP2024/054978

§ 371 Date: Aug. 26, 2025

(30) Foreign Application Priority Data
Feb. 27, 2023 (GB) 2302820.2

(51) Int. Cl. C07K 16/18; C07K 16/22; G01N 33/68

(52) U.S. Cl. CPC C07K 16/18; C07K 16/22; G01N 33/6887; G01N 2333/78; G01N 2470/10; G01N 2800/52

(57) ABSTRACT

The present invention relates to a method for detecting markers of fibroblast activation in a patient by sandwich immunoassay to identify patients, particularly cancer patients who would benefit from anti-TGFβI therapy. The markers of fibroblast activation may be selected from the N-terminal pro-peptide of Collagen III (Pro-C3), the N-terminal pro-peptide of Collagen VI (Pro-C6), the N-terminal pro-peptide of Collagen I (Pro-C1) and/or an internal sequence in the N-terminal region of type 3 collagen (P3NP). The invention also provides an anti-TGFβI therapy for use in the treatment of cancer and/or fibrosis.

### FIELD OF INVENTION

The present invention relates to a method for detecting markers of fibroblast activation in a patient by immunoassay to identify patients, particularly cancer patients who would benefit from anti-TGFβI therapy. The markers of fibroblast activation may be selected from the N-terminal pro-peptide of Collagen Ill (Pro-C3), the N-terminal pro-peptide of Collagen VI (Pro-C6), the N-terminal pro-peptide of Collagen I (Pro-C1) and/or an internal sequence in the N-terminal region of type 3 collagen (P3NP). The invention also provides an anti-TGFβI therapy for use in the treatment of cancer and/or fibrosis.

### BACKGROUND

A growing body of research has been undertaken to understand changes that occur in extracellular matrix (ECM) dynamics related to physiological impairment and lung function decline in, for example, idiopathic pulmonary fibrosis (IPF) [1-4]. In a healthy state the ECM forms a thin basement membrane layer in the small airways, separating the capillaries from the alveolar space, and allowing unimpeded gas diffusion. During fibrosis the ECM expands limiting this diffusion. The fibrotic ECM in the lungs contains high levels of type 1, Ill and VI collagens. Very simplistically, in lung fibrosis the specialised basement membrane, normally composed of an open structed type IV collagen backbone and laminin and permissive of diffusion, is replaced by a dense interstitial ECM consisting of a completely different set of collagens with, consequently, a different functionality [5].

Type Ill collagen, together with type I collagen, constitutes the major structural proteins in the human body, in which type Ill collagen is crucial for type I collagen fibrillogenesis except in bones, which almost exclusively consist of type I collagen [6, 7]. During fibrillar assembly the N-terminal propeptide of type Ill procollagen is cleaved off by specific N-proteases prior to incorporation of the mature type Ill collagen in the extracellular matrix (ECM), resulting in said N-terminal propeptide being released in the ECM and into circulation. This N-terminal propeptide molecule, also referred to as “PIIINP”, consists of three identical α-chains with a total molecular weight of 42 kDa. The removal of PIIINP is sometimes incomplete leaving the propeptide attached to the collagen molecule resulting in thin fibrils with abnormal cross-links and that are thereby prone to rapid metabolic turnover [8, 9]. Thus, this propeptide can both be a marker of formation and degradation of collagen type Ill.

WO2014/170312A1 discloses a monoclonal antibody specifically reactive with a C-terminal neo-epitope of PIIINP comprised in the C-terminal amino acid sequence CPTGXQNYSP-COOH (SEQ ID. No: 1), wherein X is Gly or Pro (said neo-epitope also being referred to herein as “Pro-C3”). It also discloses a competitive immunoassay (also referred to herein as the “Pro-C3 assay”) for detecting the levels of said C-terminal neo-epitope of PIIINP in a biological sample by contacting said sample with said monoclonal antibody, and determining the amount of binding of said antibody.

P3NP is general biomarker of fibroblast activation comprising an internal sequence in the N-terminal region of type 3 collagen with the sequence: ¹¹⁴PGIPGRNGDP¹²³ (Uniprot P02461) (SEQ ID. No: 2).

Collagen Type VI is a unique extracellular collagen which can form an independent microfibrillar network in the basement membrane of cells. It can interact with other matrix proteins including collagens, biglycan, and proteoglycans. In muscle, type VI collagen is part of the sarcolemma and is involved in anchoring the muscle fiber into the intramuscular extracellular matrix, and so is involved in force transmission. Moreover, mutations in type VI collagen can cause Bethlem myopathy and Ullrich congenital muscular dystrophy. It has been reported that the C-terminal amino acid sequence of the type VI collagen α3 chain is cleaved off from the mature type VI microfibril after secretion. However, Type VI collagen is not just involved in muscles and muscle loss.

The microflamentous interstitial type VI collagen, a triple helical molecule composed of the constituent chains α1(VI), α2(VI), and α3(VI), is expressed in most connective tissues and prominently in adipose tissue, where it anchors cells through its interconnections with other ECM proteins. During the formation of microfilaments, the triple-helical core of type VI collagen is proteolytically released from the pro-peptide, and cleavage of the C-terminal pro-peptide of the α3(VI) chain generates endotrophin, an adipokine.

PRO-C6 is a biomarker for formation of collagen type VI and endotrophin release, comprising a C-terminal epitope of the C5 domain of the α3 chain of type VI collagen that is cleaved off when a novel collagen type VI molecule assembles in the extracellular matrix, and which C-terminal epitope is also a C-terminal epitope of the bioactive fragment endotrophin. The PRO-C6 biomarker, and a PRO-C6 assay (specifically, a PRO-C6 ELISA) are described in WO2016/156526. The assay utilizes a monoclonal antibody that specifically binds to the C-terminus 10 amino acid sequence of the C5 domain of the α3 chain of collagen type VI. Endotrophin's role as a pro-fibrotic, pro-inflammatory and pro-tumorigenic molecule has been observed in preclinical models of breast cancer and liver fibrosis. PRO-C6 has been established as a prognostic biomarker for mortality and disease progression in chronic kidney disease and diabetic kidney disease patients and as a predictive marker for response to glucose lowering therapy in diabetic patients.

Type I collagen is the most abundant collagen in the body as it is the main structural protein of bone. PRO-C1 is a biomarker for formation of collagen type 1, comprising the N-terminal pro-peptide of type I collagen, having the sequence ⁹⁶PDGSESPTDQETTGV¹¹⁰ (UniProt P02452) (SEQ. ID No: 3).

Tumor fibrosis and associated extracellular matrix (ECM) and collagen synthesis is emerging as a key component in defining the abundant stromal reaction in cancer and for defining prognosis and response to anti-cancer treatment. Cancer associated fibroblasts (CAFs) are the main drivers of this so-called desmoplastic reaction and collagen synthesis. Identifying the drivers of CAF's collagen synthesis is required to optimally develop novel ambitious therapies targeting the excess deposition of ECM and collagen in the tumor microenvironment.

While the interest in pharmaceutical attenuation of fibrosis is substantial, such as in Idiopathic pulmonary fibrosis (IPF), Non-alcoholic steatohepatitis (NASH), Chronic Kidney disease (CKD) and Systemic sclerosis (SSC), the number of phase II and phase Ill failures are increasing. This may both because of lack of optimal clinical trial design and robust endpoints, but also consequent to a lack of understanding of disease drivers. Somewhat worrisome, the number of new drug targets and positive animal studies in liver and lung fibrosis are increasing, with little or no translatability to human clinical settings. There is a need to understand the pathways driving fibrosis in man. In the human clinical settings, we need to understand the common denominators as well as the fibrosis pathology divergent disease pathways for the central fibrotic indications for the benefit of patients.

The protein TGFβI (Transforming growth factor-p-induced) also known as BigH3 has previously been shown to ‘reprogram’ the tumor microenvironment in the pancreatic cancer space and associated with survival outcome which led to a suggested promising novel target of TGFβI for future anti-cancer drug development [10][11]. While the link between TGFβI, CAF activity and collagen turnover has been described [12], little is known on the direct impact of TGFβI/BigH3 on fibroblast and associated collagen synthesis.

Traditionally genome-wide association study (GWAS) analysis is made by associations to clinical parameters [13]. Clinical parameters may be considered an endpoint of diseases, whereas biochemical markers are more related to the acute disease activity. In directly alignment, PRO-C3, as measurement of type Ill collagen formation is a surrogate measurement of fibrosis formation [14]. PRO-C3 is the quantification of the pro-peptide of the type Ill collagen chain (also known as PNIIIP). When fibroblast produce type Ill collagen, the pro-peptide of the collagens is released before the collagen is incorporated into the matrix. Consequently, PRO-C3 may be considered a surrogate biomarker for the velocity of fibrosis formation. While type I collagen is the most abundant protein in the body and of the fibrotic extra cellular matrix (ECM), type Ill collagen is less abundant and more specific for the fibrotic ECM[15].

PRO-C3 levels have previously been associated with prognosis of liver fibrosis progression[16], diagnosis of liver fibrosis[15] as well as a pharmacodynamic repose to interventions which shown a reduction in fibrosis[17],[18].

The present inventors have identified the relationship between organ fibrosis, PRO-C3, PRO-C1, PRO-C6 and P3NP and the genes within pathways responsible for higher levels of fibrosis formation.

### SUMMARY

To investigate the potential relationship between the biomarker PRO-C3 and genes within pathways responsible for higher levels of fibrosis formation, GWAS analysis was carried out based on Pro-C3 levels measured using the Pro-C3 assay. A strong linkage between elevated levels of Pro-C3 and the TGFβI gene was identified. No such correlation was found between the TGFβI gene and other markers of chronic liver disease.

Given that high PRO-C3 (pro-peptide of type Ill collagen) levels, as a measure of fibrotic activity, are associated with the TGFβI risk allele rs2073511, the inventors investigated if there was a direct effect of TGFβI on fibroblasts collagen synthesis by stimulating pancreatic cancer-associated fibroblasts (CAFs) in vitro. The paralog to TGFβI is periostin which is also known to play a pivotal and multifaceted role in tumorigenesis [19]. Both these matricellular proteins contain a single emilin (EMI) and four fasciclin-1 (FAS) modules (20). Interestingly, the presence of an EMI domain on another protein (EMILIN-2) is required for a biological effect of that protein (wnt signaling/wnt1 interaction) as there was no effect with a deletion mutant lacking this domain. The EMI domain on TGFβI and periostin are closely related in share large sequence homology and generally shorter than other EMI domains (FIG. 1)[21].

The inventors have demonstrated that TGFβI causes a dose dependent increase in markers of fibroblast activation, namely Pro-C3, Pro-C1, Pro-C6 and P3NP levels. In addition, administration of the EMI domain of periostin also causes an increase in Pro-C3 levels.

Accordingly, in a first aspect, the present invention provides a method for identifying a patient who is more likely to respond to anti-TGFβI therapy, said method using an immunoassay for detecting in a patient sample at least one marker of fibroblast activation, said method comprising:
contacting said patient sample with a monoclonal antibody, wherein said monoclonal antibody is specifically reactive with an epitope of the marker of fibroblast activation;
determining the amount of binding of said antibody;
and
correlating said amount of binding with values associated with normal healthy subjects, and/or with values associated with known responders to anti-TGFβI therapy, and/or with a predetermined cut-off value.

The marker of fibroblast activation may be selected from PRO-C3 (PIIINP), PRO-C6, PRO-C1 and/or P3NP.

A high amount of binding indicates an elevated level of the marker of fibroblast activation. Elevated levels of marker of fibroblast activation indicate that a patient is more likely to respond to, and so a suitable candidate for, anti-TGFβI therapy.

The present invention provides a method for identifying a patient who is more likely to respond to anti-TGFβI therapy, said method using an immunoassay for detecting in a patient sample Pro-C3, said method comprising:
contacting said patient sample with a monoclonal antibody, wherein said monoclonal antibody specifically reactive and binds with an epitope of PIIINP;
determining the amount of binding of said antibody;
and
correlating said amount of binding with values associated with normal healthy subjects, and/or with values associated with known responders to anti-TGFβI therapy, and/or with a predetermined cut-off value.

A high amount of binding indicates an elevated level of Pro-C3. Elevated levels of Pro-C3 indicate that a patient is more likely to respond to, and so a suitable candidate for, anti-TGFβI therapy.

In preferred embodiments, the epitope of PIIINP with which the monoclonal antibody is specifically reactive and so specifically binds is a C-terminal neo-epitope of PIIINP generated by N-protease cleavage of intact type Ill procollagen. Preferably, said C-terminal neo-epitope of PIIINP is comprised in a C-terminal amino acid sequence CPTGXQNYSP-COOH (SEQ ID. No: 1) wherein X is Gly or Pro. Most preferably, the C-terminal neo-epitope of PIIINP is comprised in a C-terminal amino acid sequence CPTGPQNYSP-COOH (SEQ ID. No: 4).

Preferably, the monoclonal antibody does not specifically recognise or bind to an elongated version of said C-terminal amino acid sequence which is CPTGXQNYSPQ-COOH wherein X is Gly or Pro (SEQ ID. No: 5). Preferably, the ratio of the affinity of the antibody for amino acid sequence CPTGXQNYSP-COOH (SEQ ID. No: 1) to the affinity of said antibody for the elongated amino acid sequence CPTGXQNYSPQ-COOH (SEQ ID. No: 5) is at least 10 to 1, preferably at least 100 to 1, more preferably at least 1,000 to 1, more preferably at least 10,000 to 1, more preferably at least 100,000 to 1, and most preferably at least 1,000,000 to 1.

Preferably, the monoclonal antibody does not specifically recognise or bind to a truncated version of said C-terminal amino acid sequence which is CPTGXQNYS-COOH (SEQ ID. No: 6). Preferably, the ratio of the affinity of the monoclonal antibody for amino acid sequence CPTGXQNYSP-COOH (SEQ ID. No: 1) to the affinity of said monoclonal antibody for the truncated amino acid sequence CPTGXQNYS-COOH (SEQ ID. No: 6) is at least 10 to 1, preferably at least 100 to 1, more preferably at least 1,000 to 1, more preferably at least 10,000 to 1, more preferably at least 100,000 to 1, and most preferably at least 1,000,000 to 1.

Preferably, the monoclonal antibody is raised against a synthetic peptide comprising or consisting of the C-terminal amino acid sequence CPTGXQNYSP-COOH (SEQ ID. No: 1) wherein X is Gly or Pro. Most preferably, the synthetic peptide has the C-terminal amino acid sequence CPTGPQNYSP-COOH(SEQ ID. No: 4).

As used herein the term “neo-epitope” refers to an epitope generated on cleavage of a polypeptide. A “C-terminal neo-epitope” refers to a neo-epitope that is at the C-terminal extremity of a cleaved polypeptide, i.e. at the C-terminal end of the of the polypeptide, and is not to be construed as meaning in the general direction thereof. Likewise, “C-terminal amino acid sequence” refer to a C-terminal peptide sequence at the extremity of a polypeptide, i.e. at the C-terminal end of the of the polypeptide, and is not to be construed as meaning in the general direction thereof.

As used herein, the terms “peptide” and “polypeptide” are used synonymously.

As used herein the term “monoclonal antibody” refers to both whole antibodies and to fragments thereof that retain the binding specificity of the whole antibody, such as for example a Fab fragment, F(ab′)2 fragment, single chain Fv fragment, or other such fragments known to those skilled in the art. As is well known, whole antibodies typically have a “Y-shaped” structure of two identical pairs of polypeptide chains, each pair made up of one “light” and one “heavy” chain. The N-terminal regions of each light chain and heavy chain contain the variable region, while the C-terminal portions of each of the heavy and light chains make up the constant region. The variable region comprises three complementarity determining regions (CDRs), which are primarily responsible for antigen recognition. The constant region allows the antibody to recruit cells and molecules of the immune system.

Antibody fragments retaining binding specificity comprise at least the CDRs and sufficient parts of the rest of the variable region to retain said binding specificity.

In the methods of the present invention, monoclonal antibodies comprising any constant region known in the art can be used. Human constant light chains are classified as kappa and lambda light chains. Heavy constant chains are classified as mu, delta, gamma, alpha, or epsilon, and define the antibody's isotype as IgM, IgD, IgG, IgA, and IgE, respectively. The IgG isotype has several subclasses, including, but not limited to IgG1, IgG2, IgG3, and IgG4 in the case of humans and IgG1, IgG2a, IgG2b, IgG2c and IgG3 in the case of mice. The monoclonal antibodies may preferably be of the IgG isotype, including any one of IgG1, IgG2, IgG3 or IgG4.

The CDR of an antibody can be determined using methods known in the art such as that described by Kabat et al. Antibodies can be generated from B cell clones as described in the examples. The isotype of the antibody can be determined by ELISA specific for human or murine IgM, IgG or IgA isotype, or human IgG1, IgG2, IgG3 or IgG4 subclasses or murine IgG1, IgG2a, IgG2b, IgG2c and IgG3. The amino acid sequence of the antibodies generated can be determined using standard techniques. For example, RNA can be isolated from the cells, and used to generate cDNA by reverse transcription. The cDNA is then subjected to PCR using primers which amplify the heavy and light chains of the antibody. For example primers specific for the leader sequence for all VH (variable heavy chain) sequences can be used together with primers that bind to a sequence located in the constant region of the isotype which has been previously determined. The light chain can be amplified using primers which bind to the 3′ end of the Kappa or Lamda chain together with primers which anneal to the V kappa or V lambda leader sequence. The full length heavy and light chains can be generated and sequenced.

Monoclonal antibodies that specifically bind to the C-terminus amino acid sequence CPTGXQNYSP-COOH wherein X is Gly or Pro (SEQ ID No. 1) can be generated via any suitable techniques known in the art. For example, the monoclonal antibody may be raised against a synthetic peptide comprising or consisting of the amino acid sequence CPTGPQNYSP-COOH (SEQ ID No. 4), such as for example by: immunizing a rodent (or other suitable mammal) with a synthetic peptide consisting of the sequence CPTGPQNYSP-COOH (SEQ ID No. 4), which optionally may linked to an immunogenic carrier protein (such as keyhole limpet hemocyanin), isolating and cloning a single antibody producing cell, and assaying the resulting monoclonal antibodies to ensure that they have the desired specificity.

The term “specifically bind” as used herein means that the antibody binding is selective for the antigen and that this binding can be distinguished from unwanted or non-specific interactions. The ability of a monoclonal antibody to bind to a specific epitope or peptide sequence can be measured either through an enzyme-linked immunosorbent assay (ELISA) as described herein or other techniques familiar to one of skill in the art, e.g. surface plasmon resonance (SPR) technique (analyzed e.g. on a BIAcore instrument) and traditional binding assays. The extent of binding of a monoclonal antibody to an unrelated protein is less than about 10% of the binding of the monoclonal antibody to the epitope or peptide as measured, e.g., by ELISA. “Affinity” refers to the strength of the sum total of non-covalent interactions between a single binding site of a molecule (e.g., an epitope binding region of an antibody) and its binding partner (e.g., an epitope or antigen). Unless indicated otherwise, as used herein, “binding affinity” refers to intrinsic binding affinity which reflects a 1:1 interaction between members of a binding pair (e.g., an antigen binding moiety and an antigen). The affinity of a molecule for its partner can generally be represented by the dissociation constant (Kd), which is the ratio of dissociation and association rate constants (k_{off} and kₒₙ, respectively). Thus, equivalent affinities may comprise different rate constants, as long as the ratio of the rate constants remains the same. The dissociation constant represents the concentration of the antigen at which half of the binding sites on the antibody are occupied. A lower Kd indicates a higher binding affinity between the antibody and antigen, while a higher Kd reflects weaker binding. Several methods are available to measure the Kd of an antibody, including surface plasmon resonance (SPR), isothermal titration calorimetry (ITC), and fluorescence-based assays. In certain aspects, a monoclonal antibody that binds to the epitope or peptide has a dissociation constant (KD) of <1 pM, <100 nM, <10 nM, <1 nM, <0.1 nM, <0.01 nM, or <0.001 nM (e.g. 10⁸ M or less, e.g. from 10⁸ M to 10¹³ M, e.g., from 10⁹ M to 10¹³ M).

The monoclonal antibody that specifically binds to the PRO-C3 may preferably comprise one or more complementarity-determining regions (CDRs) selected from:

CDR-H1:
(SEQ ID. No: 7)
NYVIH
CDR-H2:
(SEQ ID. No: 8)
YMNPYNDVPKNNAKFRG
CDR-H3:
(SEQ ID. No: 9)
GGFFGPLSY
CDR-L1:
(SEQ ID. No: 10)
RSSQNIVYSNGDTYFE
CDR-L2:
(SEQ ID. No: 11)
KVSQRFS
CDR-L3:
(SEQ ID. No: 12)
FQGAHDPPA

Preferably the monoclonal antibody may comprise at least 2,3,4,5 or all 6 of the above listed CDR sequences.

Preferably the monoclonal antibody has a light chain variable region comprising the CDR sequences:

CDR-L1:
(SEQ ID. No: 10)
RSSQNIVYSNGDTYFE
CDR-L2:
(SEQ ID. No: 11)
KVSQRFS
and
CDR-L3:
(SEQ ID. No: 12)
FQGAHDPPA

Preferably the monoclonal antibody may have a light chain that comprises framework sequences between the CDRs, wherein said framework sequences are substantially identical or substantially similar to the framework sequences between the CDRs in the light chain sequence below (in which the CDRs are shown in bold and underlined, and the framework sequences are shown in italics)

(SEQ ID. No: 13)
RSSQNIVYSNGDTYFEWYLQKPGQSPKLLIYKVSQRFSGVPDRFSGSG
SGTDFTLKISRVETEDLGVYYCFQGAHDPPA

Preferably the monoclonal antibody has a heavy chain variable region comprising the CDR sequences:

CDR-H1:
(SEQ ID. No: 7)
NYVIH
CDR-H2:
(SEQ ID. No: 8)
YMNPYNDVPKNNAKFRG
and
CDR-H3:
(SEQ ID. No: 9)
GGFFGPLSY

Preferably the monoclonal antibody may have a heavy chain that comprises framework sequences between the CDRs, wherein said framework sequences are substantially identical or substantially similar to the framework sequences between the CDRs in the heavy chain sequence below (in which the CDRs are shown in bold and underlined, and the framework sequences are shown in italics)

(SEQ ID. No: 14)
NYVIHWLKQKAGQGPEWIGYMNPYNDVPKNNAKFRGKARLTSDR
SSTTAYMELNSLTSEDSAVYYCARGGFFGPLSY

As used herein, the framework amino acid sequences between the CDRs of an antibody are substantially identical or substantially similar to the framework amino acid sequences between the CDRs of another antibody if they have at least 70%, 80%, 90% or at least 95% similarity or identity. The similar or identical amino acids may be contiguous or non-contiguous.

The framework sequences may contain one or more amino acid substitutions, insertions and/or deletions. Amino acid substitutions may be conservative, by which it is meant the substituted amino acid has similar chemical properties to the original amino acid. A skilled person would understand which amino acids share similar chemical properties. For example, the following groups of amino acids share similar chemical properties such as size, charge and polarity: Group 1 Ala, Ser, Thr, Pro, Gly; Group 2 Asp, Asn, Glu, Gln; Group 3 His, Arg, Lys; Group 4 Met, Leu, lie, Val, Cys; Group 5 Phe Thy Trp.

A program such as the CLUSTAL program to can be used to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment.

A program like BLASTx will align the longest stretch of similar sequences and assign a value to the fit. It is thus possible to obtain a comparison where several regions of similarity are found, each having a different score. Both types of analysis are contemplated in the present invention. Identity or similarity is preferably calculated over the entire length of the framework sequences.

In certain preferred embodiments, the monoclonal antibody that specifically binds to the PRO-C3 may comprise the light chain variable region sequence:

(SEQ ID. No: 15)
DVLMTQTPLSLSVSLGDQASISCRSSQNIVYSNGDTYFEW
YLQKPGQSPKLLIYKVSQRFSGVPDRFSGSGSGTDFTLKI
SRVETEDLGVYYC FQGAHDPPAFGGGTKLELK

and/or the heavy chain variable region sequence:

(SEQ ID. No: 16)
EVQLQQSGPEVLKPGASVKMSCKASGYTFINYVIHWLKQKAGQGPE
WIGYMNPYNDVPKNNAKFRGKARLTSDRSSTTAYMELNSLTSEDSA
VYYCARGGFFGPLSYWGQGTLVTVSA

(CDRs bold and underlined; Framework sequences in italics) Suitable immunoassay kits for measuring the levels of Pro-C3 are commercially available such as that from Nordic Biosciences (cat #1700AF06).

The present invention provides a method for identifying a patient who is more likely to respond to anti-TGFβI therapy, said method using an immunoassay for detecting in a patient sample Pro-C6, said method comprising:
contacting said patient sample with a monoclonal antibody, wherein said monoclonal antibody is specifically reactive with an C-terminal epitope of the C5 domain of the α3 chain of type VI collagen;
determining the amount of binding of said antibody;
and
correlating said amount of binding with values associated with normal healthy subjects, and/or with values associated with known responders to anti-TGFβI therapy, and/or with a predetermined cut-off value.

A high amount of binding indicates an elevated level of Pro-C6. Elevated levels of Pro-C6 indicate that a patient is more likely to respond to, and so a suitable candidate for, anti-TGFβI therapy.

Preferably the monoclonal antibody which is specifically reactive and so specifically binds the C-terminal epitope of the C5 domain of the α3 chain of type VI collagen specifically binds to the C-terminus amino acid sequence KPGVISVMGT (SEQ ID No: 17) (also referred to herein as the “PRO-C6 sequence”, or simply “PRO-C6”). Preferably said monoclonal antibody does not specifically bind to an elongated version of said C-terminus amino acid sequence which is KPGVISVMGTA (SEQ ID No: 18), or to a truncated version of said C-terminus amino acid sequence which is KPGVISVMG (SEQ ID No: 19).

Preferably, the ratio of the affinity of said antibody for the C-terminus amino acid sequence KPGVISVMGT (SEQ ID No: 17) to the affinity of said antibody for the elongated C-terminus amino acid sequence KPGVISVMGTA (SEQ ID No: 18), and/or for the truncated C-terminus amino acid sequence KPGVISVMG (SEQ ID No: 19), is at least 10 to 1, and more preferably is at least 50 to 1, at least 100 to 1, at least 500 to 1, at least 1,000 to 1, at least 10,000 to 1, at least 100,000 to 1, or at least 1,000,000 to 1.

Monoclonal antibodies that specifically bind to the C-terminus amino acid sequence KPGVISVMGT (SEQ ID No. 17) can be generated via any suitable techniques known in the art. For example, the monoclonal antibody may be raised against a synthetic peptide comprising or consisting of the amino acid sequence KPGVISVMGT (SEQ ID No. 17), such as for example by: immunizing a rodent (or other suitable mammal) with a synthetic peptide consisting of the sequence KPGVISVMGT (SEQ ID No. 17), which optionally may linked to an immunogenic carrier protein (such as keyhole limpet hemocyanin), isolating and cloning a single antibody producing cell, and assaying the resulting monoclonal antibodies to ensure that they have the desired specificity.

The monoclonal antibody that specifically binds to the PRO-C6 sequence may preferably comprises one or more complementarity-determining regions (CDRs) selected from:

CDR-L1:
(SEQ ID No: 20)
RSSQRIVHSNGITFLE
CDR-L2:
(SEQ ID No: 21)
RVSNRFS
CDR-L3:
(SEQ ID No: 22)
FQGSHVPLT
CDR-H1:
(SEQ ID No: 23)
DFNMN
CDR-H2:
(SEQ ID No: 24)
AINPHNGATSYNQKFSG
CDR-H3:
(SEQ ID No: 25)
WGNGKNS.

Preferably the antibody comprises at least 2,3,4,5 or 6 of the above listed CDR sequences.

Preferably the monoclonal antibody light chain variable region comprises the CDR sequences

CDR-L1:
(SEQ ID No: 20)
RSSQRIVHSNGITFLE
CDR-L2:
(SEQ ID No: 21)
RVSNRFS
and
CDR-L3:
(SEQ ID No: 22)
FQGSHVPLT.

Preferably the monoclonal antibody light chain comprises framework sequences between the CDRs, wherein said framework sequences are substantially identical or substantially similar to the framework sequences between the CDRs in the light chain sequence below (in which the CDRs are shown in bold and underlined, and the framework sequences are shown in italics)

(SEQ ID NO: 26)
RSSQRIVHSNGITFLEWYLQKPGQSPKLLIYRVSNRFSGVPDR
FSGSGSGTDFTLKISRVEAEDLGLYYCFQGSHVPLT

Preferably the monoclonal antibody heavy chain variable region comprises the CDR sequences:

CDR-H1:
(SEQ ID No: 23)
DFNMN
CDR-H2:
(SEQ ID No: 24)
AINPHNGATSYNQKFSG
and
CDR-H3:
(SEQ ID No: 25)
WGNGKNS.

Preferably the monoclonal antibody heavy chain comprises framework sequences between the CDRs, wherein said framework sequences are substantially identical or substantially similar to the framework sequences between the CDRs in the heavy chain sequence below (in which the CDRs are shown in bold and underlined, and the framework sequences are shown in italics)

(SEQ ID NO: 27)
DFNMNWVKQSHGKSLEWIGAINPHNGATSYNQKFSGKATLTVDKSSS
TAYMELNSLTSDDSAVYYCARWGNGKNS

In certain preferred embodiments, the monoclonal antibody that specifically binds to the PRO-C6 sequence may comprise the light chain variable region sequence:

(SEQ ID No: 28)
DVVMTQTPLSLPVNLGDQASISCRSSQRIVHSNGITFLEWYLQKP
GQSPKLLIYRVSNRFSGVPDRFSGSGSGTDFTLKISRVEAEDLGL
YYCFQGSHVPLTFGAGTRLELK

and/or the heavy chain variable region sequence:

(SEQ ID NO: 29)
EVQLQQSGPVMVKPGTSVKTSCKASGYTFTDFNMNWVKQSHGK
SLEWIGAINPHNGATSYNQKFSGKATLTVDKSSSTAYMELNSL
TSDDSAVYYCARWGNGKNSWGQGTTLTVSS

(CDRs bold and underlined; Framework sequences in italics) Suitable immunoassay kits for measuring the levels of Pro-C6 are commercially available such as that from Nordic Biosciences (cat #4000AF02).

The present invention provides a method for identifying a patient who is more likely to respond to anti-TGFβI therapy, said method using an immunoassay for detecting in a patient sample Pro-C1, said method comprising:
contacting said patient sample with a monoclonal antibody, wherein said monoclonal antibody is specifically reactive with an N-terminal epitope of the pro-peptide of collagen type I;
determining the amount of binding of said antibody;
and
correlating said amount of binding with values associated with normal healthy subjects, and/or with values associated with known responders to anti-TGFβI therapy, and/or with a predetermined cut-off value.

A high amount of binding indicates an elevated level of Pro-C1. Elevated levels of Pro-C1 indicate that a patient is more likely to respond to, and so a suitable candidate for, anti-TGFβI therapy.

Preferably the monoclonal antibody which is specifically reactive and so specifically binds the N-terminal epitope of the pro-peptide of collagen type I specifically binds to the N-terminal amino acid sequence PDGSESPTDQETTGV (SEQ ID No: 3) (also referred to herein as the “PRO-C1 sequence”, or simply “PRO-C1”). Preferably said monoclonal antibody does not specifically bind to an elongated version of said N-terminus amino acid sequence which is PDGSESPTDQETTGVE (SEQ ID No: 30), or to a truncated version of said N-terminus amino acid sequence which is DGSESPTDQETTGV (SEQ ID No: 31).

Preferably, the ratio of the affinity of said antibody for the N-terminus amino acid sequence PDGSESPTDQETTGV (SEQ ID No: 3) to the affinity of said antibody for the elongated N-terminus amino acid sequence PDGSESPTDQETTGVE (SEQ ID No: 30), and/or for the truncated N-terminus amino acid sequence DGSESPTDQETTGV (SEQ ID No: 31), is at least 10 to 1, and more preferably is at least 50 to 1, at least 100 to 1, at least 500 to 1, at least 1,000 to 1, at least 10,000 to 1, at least 100,000 to 1, or at least 1,000,000 to 1.

As used herein the term “N-terminus” refers to a N-terminal peptide sequence at the extremity of a polypeptide, i.e. at the N-terminal end of the polypeptide, and is not to be construed as meaning in the general direction thereof.

Monoclonal antibodies that specifically bind to the N-terminus amino acid sequence PDGSESPTDQETTGV (SEQ ID No. 3) can be generated via any suitable techniques known in the art. For example, the monoclonal antibody may be raised against a synthetic peptide comprising or consisting of the amino acid sequence PDGSESPTDQETTGV (SEQ ID No. 3), such as for example by: immunizing a rodent (or other suitable mammal) with a synthetic peptide consisting of the sequence PDGSESPTDQETTGV (SEQ ID No. 3), which optionally may linked to an immunogenic carrier protein (such as keyhole limpet hemocyanin), isolating and cloning a single antibody producing cell, and assaying the resulting monoclonal antibodies to ensure that they have the desired specificity.

Suitable immunoassay kits for measuring the levels of Pro-C1 are commercially available such as that from Nordic Biosciences (cat #2800A0E51).

The present invention provides a method for identifying a patient who is more likely to respond to anti-TGFβI therapy, said method using an immunoassay for detecting in a patient sample P3NP, said method comprising:
contacting said patient sample with a monoclonal antibody, wherein said monoclonal antibody is specifically reactive with an internal sequence in the N-terminal region of type 3 collagen;
determining the amount of binding of said antibody;
and
correlating said amount of binding with values associated with normal healthy subjects, and/or with values associated with known responders to anti-TGFβI therapy, and/or with a predetermined cut-off value.

A high amount of binding indicates an elevated level of P3NP. Elevated levels of P3NP indicate that a patient is more likely to respond to, and so a suitable candidate for, anti-TGFβI therapy.

Preferably the monoclonal antibody which is specifically reactive and so specifically binds an internal sequence in the N-terminal region of type 3 collagen specifically binds to the amino acid sequence PGIPGRNGDP (SEQ ID No: 2) (also referred to herein as the “P3NP sequence”, or simply “P3NP”). Preferably said monoclonal antibody does not specifically bind to an elongated version of said amino acid sequence which is PPGIPGRNGDP (SEQ ID No: 32) or PGIPGRNGDPG (SEQ ID No: 33), or to a truncated version of said N-terminus amino acid sequence which is GIPGRNGDP (SEQ ID No: 34) or PGIPGRNGD (SEQ ID No: 35).

Preferably, the ratio of the affinity of said antibody for the PGIPGRNGDP (SEQ ID No: 2) to the affinity of said antibody for the elongated amino acid sequence PPGIPGRNGDP (SEQ ID No: 32) or PGIPGRNGDPG (SEQ ID No: 33), and/or for the truncated amino acid sequence GIPGRNGDP (SEQ ID No: 34) or PGIPGRNGD (SEQ ID No: 35), is at least 10 to 1, and more preferably is at least 50 to 1, at least 100 to 1, at least 500 to 1, at least 1,000 to 1, at least 10,000 to 1, at least 100,000 to 1, or at least 1,000,000 to 1.

Monoclonal antibodies that specifically bind to the amino acid sequence PGIPGRNGDP (SEQ ID No. 2) can be generated via any suitable techniques known in the art. For example, the monoclonal antibody may be raised against a synthetic peptide comprising or consisting of the amino acid sequence PGIPGRNGDP (SEQ ID No. 2), such as for example by: immunizing a rodent (or other suitable mammal) with a synthetic peptide consisting of the sequence PGIPGRNGDP (SEQ ID No. 2), which optionally may linked to an immunogenic carrier protein (such as keyhole limpet hemocyanin), isolating and cloning a single antibody producing cell, and assaying the resulting monoclonal antibodies to ensure that they have the desired specificity.

Suitable immunoassay kits for measuring the levels of P3NP are commercially available such as that from Nordic Biosciences (cat #1090BD01).

The method may measure the level of one, two, three, or four markers of fibroblast activation. The method may measure the level of Pro-C3, and optionally one, two or three other markers of fibroblast activation. The method may measure the level of Pro-C3 and Pro-C6, Pro-C3 and Pro-C1 or Pro-C3 and P3NP. The method may measure the level of Pro-C3, Pro-C6, and Pro-C1 or Pro-C3, Pro-C6, and P3NP.

The method may measure the level of Pro-C6, and optionally one, two or three other markers of fibroblast activation. The method may measure the level of Pro-C6 and Pro-C1 or Pro-C6 and P3NP. The method may measure the level of Pro-C6, Pro-C1 and P3NP.

The method may measure the level of Pro-C1, and optionally one, two or three other markers of fibroblast activation. The method may measure the level of Pro-C1 and P3NP.

The method may measure the level of P3NP, and optionally one, two or three other markers of fibroblast activation.

The method may measure the level of Pro-C3, Pro-C6, Pro-C1 and P3NP.

In preferred embodiments, the patient sample is a biofluid. The biofluid may be, but not limited to, serum, plasma, urine, amniotic fluid, tissue supernatant or cell supernatant. Preferably the biofluid is blood, serum or plasma.

The immunoassay may be, but is not limited to, a competition assay or a sandwich assay. The immunoassay may, for example, be a radioimmunoassay or an enzyme-linked immunosorbent assay (ELISA). Such assays are techniques known to the person skilled in the art.

In preferred embodiments, the immunoassay is a competition immunoassay. In preferred embodiments, the competition immunoassay is a radioimmunoassay, fluorescence immunoassay, or an enzyme-linked immunosorbent assay (ELISA). Competition immunoassays, including competition immunoassays carried out as radioimmunoassays, fluorescence immunoassays, or ELISAs, are methods and techniques that are well known to the person skilled in the art.

In preferred embodiments, the immunoassay is a sandwich immunoassay. In preferred embodiments, the sandwich immunoassay is a radioimmunoassay, fluorescence immunoassay, or an enzyme-linked immunosorbent assay (ELISA). Sandwich immunoassays, including sandwich immunoassays carried out as radioimmunoassays, fluorescence immunoassays, or ELISAs, are methods and techniques that are well known to the person skilled in the art. As is well known, in a sandwich immunoassay at least two antibodies are used for the detection of an antigen in a sample, with at least one of the antibodies acting as a catcher antibody and typically being bound to a solid support, and at least one other antibody acting as a detection antibody. In the case of the present invention the monoclonal antibody may function as a catcher antibody or a detection antibody.

As noted above, the monoclonal antibody may be bound to a solid support. Any suitable form of solid support and binding method known in the art can be used. For example, in certainly exemplary embodiments the monoclonal antibody may be biotinylated and bound to a streptavidin-coated solid support.

In alternative embodiments, the monoclonal antibody may be labelled to allow detection of the monoclonal antibody and determination of the amount of binding between the monoclonal antibody and marker of fibroblast activation such as PRO-C3, PRO-C6, PRO-C1 or P3NP bound to a first further monoclonal antibody on the solid support.

For example, in one preferred embodiment the monoclonal antibody may be an enzyme-linked antibody. The enzyme may be, but is not limited to, horseradish peroxidase (HRP).

In another preferred embodiment the monoclonal antibody may be radiolabelled or linked to a fluorophore.

Although these are preferred labels to be used with the invention, it is envisaged that any suitable labelling system may be employed, such as, but not limited to, DNA reporters or electro-chemiluminescent tags.

Alternatively, a second further antibody which is labelled and which recognises the monoclonal antibody may be used to detect the monoclonal antibody and determine the amount of binding between the second monoclonal antibody and marker of fibroblast activation bound to the first further monoclonal antibody on the solid support. The second further labelled antibody may be labelled using a label as described above.

As used herein the term “amount of binding” refers to the quantification of binding between the monoclonal antibody and marker of fibroblast activation such as PRO-C3 (PIIINP), PRO-C6, PRO-C1 or P3NP from the sample. Said quantification may for example be determined by comparing the measured values of binding of the marker of fibroblast activation from the patient sample against a calibration curve produced using measured values of binding of marker of fibroblast activation from standard samples comprising supernatant from a “Scar-in-a-jar” (SiaJ) model in which healthy primary human lung fibroblasts were cultured in the presence of ficoll and transforming growth factor-beta (TGF-β), in order to thereby determine the quantity of marker of fibroblast activation in the patient sample. The marker of fibroblast activation may be selected from PRO-C3 (PIIINP), PRO-C6, PRO-C1 and/or P3NP. In the Examples set out below, an ELISA method is used in which spectrophotometric analysis is used to measure the amount of binding both from the patient samples and when producing the calibration curve. However, any suitable analytical method can be used.

As used herein the term “predetermined cut-off value” means an amount of binding that is determined statistically to be indicative of a high likelihood of a patient who will respond to anti-TGFβI therapy, in that a measured value of the target peptide in a patient sample that is at or above the statistical cut-off value corresponds to at least a 70% probability, preferably at least an 75% probability, more preferably at least an 80% probability, more preferably at least an 85% probability, more preferably at least a 90% probability, and most preferably at least a 95% probability of the patient being successfully treated with anti-TGFβI therapy.

As used herein, the term “values associated with normal healthy subjects” means standardised quantities of binding determined by the method described supra for samples from subjects considered to be healthy, i.e. without disease for example those without cancer or the specific cancer of interest; and the term “values associated with known responders to anti-TGFβI therapy” means standardised quantities of binding determined by the method described supra for samples from patients known to have responded positively to anti-TGFβI therapy i.e. those who have significantly improved following treatment with an anti-TGFβI agent. A “significant improvement” may be a reduction or elimination of symptoms, regression of a tumour, improved prognosis or change in biomarkers indicative of disease severity which indicate as reduction in disease severity.

Accordingly, in these embodiments the method may further comprise administering anti-TGFβI therapy to patient identified as likely to respond to anti-TGFβI therapy. Suitable therapy includes administration of a neutralising antibody which targets TGFβI.

The patient is preferably a subject suffering from cancer and/or fibrosis. The cancer may be bladder cancer, breast cancer, colorectal cancer, head and neck cancer, kidney cancer, liver cancer, lung cancer, melanoma, ovarian cancer, pancreatic cancer, prostate cancer, or stomach cancer. The cancer may be stage I, Stage II, stage Ill, or stage IV. The tumour may be a primary tumour or metastatic.

In a second aspect the present invention provides an agent which binds to the EMI domain of TGFβI or periostin or an analogue thereof.

The “EMI domain” as used herein refers to EMI domain of periostin or TGFβI or an analogue thereof. Specifically, the “EMI domain” refers to 56 amino acid EMI-domain from Periostin (bases 40-94 of the protein sequence) or the 56 amino acid EMI-domain from TGFβI (bases 45-99 of the protein sequence). Preferably the EMI-domain from Periostin comprises or consists essentially of:

(SEQ ID No. 36)
GPNVCALQQILGTKKKYFSTCKNWYKKSICGQKTTVLYE
CCPGYMRMEGMKGCPA.

Preferably the EMI-domain from TGFβI comprises or consists essentially of:

(SEQ ID No. 37)
GPNVCAVQKVIGTNRKYFTNCKQWYQRKICGKSTVISYECC
PGYEKVPGEKGCPA.

Analogues of the EMI domain for use in the present invention may include proteins comprising a sequence similar to the amino acid sequence defined in SEQ ID No. 36 or SEQ ID No. 37, and which have the same biological effect i.e. increase marker of fibroblast activation levels, for example increase Pro-C3, Pro-C6, Pro-C1 and/or P3NP levels. For example, the EMI domain analogue may have at least 70%, 80%, 90% or at least 95% similarity to the sequence defined in SEQ ID No. 36 or SEQ ID No. 37 or a fragment of the sequence defined by SEQ ID No. 36 or SEQ ID No. 37. Alternatively, the EMI domain analogue may have at least 70%, 80%, 90% or at least 95% identity to the sequence defined in SEQ ID No. 36 or SEQ ID No. 37 or a fragment of the sequence defined by SEQ ID No. 36 or SEQ ID No. 37. For example, the EMI domain analogue may comprise an amino acid sequence that is identical to 50, 51, 52, 53, 54, 55, 56 or 57 amino acids of the sequence defined by SEQ ID No. 36 or SEQ ID No. 37. The similar or identical amino acids may be contiguous or non-contiguous.

A program such as the CLUSTAL program to can be used to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of analysis are contemplated in the present invention. Identity or similarity is preferably calculated over the entire length of SEQ ID No. 36 or SEQ ID No. 37.

The analogue of EMI domain may contain one or more amino acid substitutions, insertions and/or deletions.

Amino acid substitution means that an amino acid residue is substituted for a replacement amino acid residue at the same position. Amino acid substitutions may be conservative, by which it is meant the substituted amino acid has similar chemical properties to the original amino acid. A skilled person would understand which amino acids share similar chemical properties. For example, the following groups of amino acids share similar chemical properties such as size, charge and polarity: Group 1 Ala, Ser, Thr, Pro, Gly; Group 2 Asp, Asn, Glu, Gln; Group 3 His, Arg, Lys; Group 4 Met, Leu, lie, Val, Cys; Group 5 Phe Thy Trp.

Inserted amino acid residues may be inserted at any position and may be inserted such that some or all of the inserted amino acid residues are immediately adjacent one another or may be inserted such that none of the inserted amino acid residues is immediately adjacent another inserted amino acid residue. For example, the EMI domain analogue may comprise 1, 2, 3, 4, 5, 6, 7, 8, 9 or 10 additional amino acids at the N- and/or C-terminus of the amino acid sequence defined in SEQ ID No. 36 or SEQ ID No. 37.

One, two or three amino acids may be deleted from the sequence of SEQ ID No. 36 or SEQ ID No. 37. Each deletion can take place at any position of SEQ ID No. 36 or SEQ ID No. 37.

Inserted amino acids and replacement amino acids may be naturally occurring amino acids or may be non-naturally occurring amino acids and, for example, may contain a non-natural side chain, and/or be linked together via non-native peptide bonds. Such altered peptide ligands are known in the art. If more than one amino acid residue is substituted and/or inserted, the replacement/inserted amino acid residues may be the same as each other or different from one another. Each replacement amino acid may have a different side chain to the amino acid being replaced.

Analogues of the EMI domain may comprise one or more modified bases, wherein the amino acid residues may be chemically modified. Examples of chemical modifications include those corresponding to post translational modifications for example phosphorylation, acetylation and deamidation. Chemical modifications may not correspond to those that may be present in vivo. For example, the N or C terminal ends of the EMI domain peptide may be modified improve the stability, bioavailability and or affinity of the peptides. Further examples of non-natural modifications include incorporation of non-encoded α-amino acids, photoreactive cross-linking amino acids, N-methylated amino acids, and β-amino acids, backbone reduction, retroinversion by using d-amino acids, N-terminal methylation and C-terminal amidation and pegylation.

Preferably the agent is an antibody that specifically binds to the EMI domain or an analogue thereof. Preferably, the antibody specifically binds to the EMI domain active site.

The present invention relates to a method of producing a monoclonal antibody that specifically binds to the amino acid sequence of the EMI domain comprising raising the monoclonal antibody against a synthetic peptide comprising or consisting of an amino acid sequence of the EMI domain.

The synthetic peptide may comprise 5-15 continuous amino acids of the EMI domain. The synthetic peptide may comprise or consist of a sequence of amino acids shared by the periostin and TGFβI EMI domain. The synthetic peptide may comprise or consist of a sequence of amino acids which form part of the periostin or TGFβI EMI domain from a region which has a high percentage of homology between the two sequences, i.e a region where the amino acid sequence of both the periostin and TGFβI EMI domain is highly similar or identical. Preferably the synthetic peptide has at least 70%, 80%, 90% or at least 95% similarity or identity to the periostin and/or TGFβI EMI domain. The similar or identical amino acids may be contiguous or non-contiguous. Preferably the synthetic peptide has 100% similarity or identity to the periostin and/or TGFβI EMI domain.

Preferably, the synthetic peptide comprises or consists of the EMI domain active sites.

Preferably the synthetic peptide comprises GPNVCAX₁Q (SEQ ID NO: 38) where X₁ is V or L or YECCPGY (SEQ ID NO: 39), or GX₂KGCPA (SEQ ID NO: 40) where X₂ is E or M.

The method may, for example, comprise immunizing a non-human mammal with a synthetic peptide comprising or consisting of an amino acid sequence of the EMI domain, isolating a spleen cell from the immunized mammal that produces an antibody that specifically binds to the amino acid sequence of the EMI domain, fusing the isolated spleen cell with a hybridoma cell, and culturing the resultant hybridoma cells to secure monoclonal growth.

It is preferred that the synthetic peptide is linked (via a linker of any suitable type) to an immunogenic carrier protein.

The present invention also relates to a method of screening for an antibody that specifically binds to the amino acid sequence of the EMI domain comprising contacting a sample containing one or more antibodies with a synthetic peptide comprising or consisting of the EMI domain, and detecting binding between the antibody and the peptide.

In a preferred embodiment, the synthetic peptide is a peptide comprising or consisting of the amino acid sequence of the EMI domain, which peptide is linked (via a linker of any suitable type) to a solid support and/or to a molecule (such as for example biotin) allowing attachment to a solid support. In a particularly preferred embodiment, the synthetic peptide is a peptide consisting of the amino acid sequence of the EMI domain which peptide is linked (via a linker of any suitable type) to a solid support and/or to a molecule (such as for example biotin) allowing attachment to a solid support.

In a preferred embodiment, binding between the antibody and the peptide is detected via an enzyme-linked immunosorbent assay (ELISA).

In a preferred embodiment, the hybridoma cell that produces an antibody that specifically binds to the amino acid sequence of the EMI domain is produced via a method that comprises immunizing a non-human mammal with a synthetic peptide comprising or consisting of the amino acid sequence of the EMI domain, isolating a spleen cell from the immunized mammal that produces an antibody that specifically binds to the amino acid sequence of the EMI domain, fusing the isolated spleen cell with a hybridoma cell, and culturing the resultant hybridoma cells to secure monoclonal growth.

The present invention relates to a monoclonal antibody that that specifically binds to the amino acid sequence of the EMI domain, preferably the sequence of SEQ ID NO: 1 or SEQ ID NO: 2. Preferably, the monoclonal antibody is a monoclonal antibody that has been raised against a synthetic peptide comprising or consisting of the amino acid sequence the EMI domain.

The present invention relates to a method of producing a monoclonal antibody that specifically binds to the amino acid sequence GPNVCAX₁Q (SEQ ID NO: 38) where X₁ is V or L or YECCPGY (SEQ ID NO: 39), or GX₂KGCPA (SEQ ID NO: 40) where X₂ is E or M comprising raising the monoclonal antibody against a synthetic peptide comprising or consisting of the amino acid GPNVCAX₁Q (SEQ ID NO: 38), YECCPGY (SEQ ID NO: 39), or GX₂KGCPA (SEQ ID NO: 40).

The method may, for example, comprise immunizing a non-human mammal with a synthetic peptide comprising or consisting of the amino acid sequence GPNVCAX₁Q (SEQ ID NO: 38), and/or YECCPGY (SEQ ID NO: 39), and/or GX₂KGCPA (SEQ ID NO: 40), isolating a spleen cell from the immunized mammal that produces an antibody that specifically binds to the amino acid sequence GPNVCAX₁Q (SEQ ID NO: 38), and/or YECCPGY (SEQ ID NO: 39), and/or GX₂KGCPA (SEQ ID NO: 40), fusing the isolated spleen cell with a hybridoma cell, and culturing the resultant hybridoma cells to secure monoclonal growth.

It is preferred that the synthetic peptide is a peptide comprising or consisting of the amino acid sequence GPNVCAX₁Q (SEQ ID NO: 38), and/or YECCPGY (SEQ ID NO: 39), and/or GX₂KGCPA (SEQ ID NO: 40) which peptide is linked (via a linker of any suitable type) to an immunogenic carrier protein.

The present invention also relates to a method of screening for an antibody that specifically binds to the amino acid sequence GPNVCAX₁Q (SEQ ID NO: 38), and/or YECCPGY (SEQ ID NO: 4), and/or GX₂KGCPA (SEQ ID NO: 40) comprising contacting a sample containing one or more antibodies with a synthetic peptide according to the first aspect, and detecting binding between the antibody and the peptide.

In a preferred embodiment, the synthetic peptide is a peptide comprising or consisting of the amino acid sequence GPNVCAX₁Q (SEQ ID NO: 38), and/or YECCPGY (SEQ ID NO: 39), and/or GX₂KGCPA (SEQ ID NO: 40), which peptide is linked (via a linker of any suitable type) to a solid support and/or to a molecule (such as for example biotin) allowing attachment to a solid support. In a particularly preferred embodiment the synthetic peptide is a peptide consisting of the amino acid sequence GPNVCAX₁Q (SEQ ID NO: 38), and/or YECCPGY (SEQ ID NO: 39), and/or GX₂KGCPA (SEQ ID NO: 40) which peptide is linked (via a linker of any suitable type) to a solid support and/or to a molecule (such as for example biotin) allowing attachment to a solid support.

In a preferred embodiment, binding between the antibody and the peptide is detected via an enzyme-linked immunosorbent assay (ELISA).

In a preferred embodiment, the hybridoma cell that produces an antibody that specifically binds to the amino acid sequence GPNVCAX₁Q (SEQ ID NO: 38), and/or YECCPGY (SEQ ID NO: 39), and/or GX₂KGCPA (SEQ ID NO: 40) is produced via a method that comprises immunizing a non-human mammal with a synthetic peptide comprising or consisting of the amino acid sequence GPNVCAX₁Q (SEQ ID NO: 38), and/or YECCPGY (SEQ ID NO: 39), and/or GX₂KGCPA (SEQ ID NO: 40), isolating a spleen cell from the immunized mammal that produces an antibody that specifically binds to the amino acid sequence GPNVCAX₁Q (SEQ ID NO: 38), and/or YECCPGY (SEQ ID NO: 39), and/or GX₂KGCPA (SEQ ID NO: 40), fusing the isolated spleen cell with a hybridoma cell, and culturing the resultant hybridoma cells to secure monoclonal growth.

The present invention relates to a monoclonal antibody that that specifically binds to the amino acid sequence GPNVCAX₁Q (SEQ ID NO: 38), and/or YECCPGY (SEQ ID NO: 39), and/or GX₂KGCPA (SEQ ID NO: 40) Preferably, the monoclonal antibody is a monoclonal antibody that has been raised against a synthetic peptide comprising or 5 consisting of the amino acid sequence GPNVCAX₁Q (SEQ ID NO: 38), and/or YECCPGY (SEQ ID NO: 39), and/or GX₂KGCPA (SEQ ID NO: 40).

The monoclonal antibodies may be produced by raising the monoclonal antibody against a synthetic peptide comprising or consisting of the EMI domain active sites. The monoclonal antibodies can be raised via suitable techniques known those skilled in the art such as, but not limited to, immunizing a mouse or other non-human mammal, isolating and fusing spleen cells (B-cells) from the immunized mammal with hybridoma cells, and then culturing the resultant hybridoma cells to secure monoclonal growth.

For example, monoclonal antibodies can be generated from B cell clones as described in the examples. Preferably the monoclonal antibodies are humanized antibodies comprising human framework and/or or constant region sequences. Such antibodies may, for example, by produced from a mouse (or other non-human mammal) transgenic for human immunoglobulin genes, or by generating chimeric antibodies whereby the a mouse (or other non-human mammal) monoclonal antibody is first produced, following which the antigen binding sequences (comprising at least the CDRs) of said antibody are determined, and then a recombinant antibody is produced comprising said antibody binding sequences grafted to human framework and/or constant domain sequences.

Also provided herein are methods of screening for an antibody that specifically binds to the EMI domain active site.

The methods of screening for an antibody that specifically binds to the EMI domain active site may comprise contacting a sample containing one or more antibodies with a synthetic peptide comprising or consisting of the EMI domain active site, and detecting binding between the antibody and the peptide.

Binding may be detected via an enzyme-linked immunosorbent assay (ELISA). Various suitable forms of ELISA are known in the art and may suitably be used. For example, an ELISA technique as described in the examples may be used, whereby a fluid sample containing one or more antibodies is brought into contact with a solid support to which a peptide comprising or consisting of the EMI domain site is bound, so that binding between the bound peptide and any antibodies specific for the EMI domain active site can take place.

Following removal of any unbound antibodies, an enzyme-linked antibody can then be added that will bind to any antibodies that are bound, via the peptide comprising or consisting of the EMI domain active site, to the solid support. The activity of the enzyme can then be assessed by incubation with a substrate generating a measurable product, thereby allowing quantitative detection of any antibodies bound to the peptide comprising or consisting of the EMI domain active site that is bound to the solid support.

The above-described monoclonal antibodies that target the EMI domain 2 active site, can inhibit the fibrogenic effects of TGFβI by binding to and blocking access to the EMI active site (thereby abrogating fibrosis elicited by TGFβI). In addition to this direct neutralizing effect, those monoclonal antibodies that have an Fc region can also act to abrogate fibrosis via Fc mediated clearance of antibody bound EMI/TGFβI. Thus, the antibodies can be used for the treatment of fibrotic diseases and pathological fibrosis in general.

However, monoclonal antibodies that target other portions of the EMI can also be used to abrogate the effects of TGFβI. In particular, even though such antibodies might not block access to the EMI active sites, such antibodies can be used to effect Fc mediated clearance of TGFβI.

In a third aspect the present invention provides an agent of the second aspect for use in a method of treating cancer and/or fibrosis. Thus, the invention provides an agent which binds to the EMI domain of TGFβI or an analogue thereof for use in the treatment of cancer. In a preferred embodiment, the present invention relates to a monoclonal antibody that specifically binds to the EMI domain or an analogue thereof, for use in the treatment of cancer.

The cancer may be bladder cancer, breast cancer, colorectal cancer, head and neck cancer, kidney cancer, liver cancer, lung cancer, melanoma, ovarian cancer, pancreatic cancer, prostate cancer, or stomach cancer. The cancer may be stage I, Stage II, stage Ill, or stage IV. The tumour may be a primary tumour or metastatic.

Preferably the agent is used to treat a subject who has been identified as being more likely to be responsive to anti-TGFβI treatment. In a more preferred embodiment, the subject has elevated levels of a marker of fibroblast activation as identified by a method of the first aspect and described herein. The marker of fibroblast activation may be selected from Pro-C3, Pro-C6, Pro-C1 and/or P3NP

In a further aspect the invention provides an agent which binds to the EMI domain of TGFβI or an analogue thereof for use as a medicament. In a preferred embodiment, the present invention relates to a monoclonal antibody that specifically binds to the EMI domain or an analogue thereof, for use as a medicament.

### FIGURES

FIG. 1: (a) shows multiple sequence alignment of EMI domains. Highlighted in box are periostin (POSTN_40-94) and TGFβI (TGFβI_40-99)
(b) Amino acid sequence of EMI domains from
TGFβI (h bases 40-94) and
Periostin (horg/upot/Q15_63 bases 40-99)

FIG. 2: shows a Manhattan plot of genome-wide associations with log 2(PRO-C3) levels and 8 other liver-related biomarkers and scores. Optimized linear additive regression model was performed on 4968 PERF subjects corrected for baseline age, 3 lead principal components, serum and blood biochemistry parameters identified by elastic net and PRO-C3 measurement batch. Linear additive regression model adjusted for baseline age and the 3 leading principal components was performed on ADAPT, FIB4, APRI and NFS disease scores, as well as log 2-transformed levels of liver enzymes alanine aminotransferase (ALT), aspartate aminotransferase (AST), alkaline phosphatase (AP) and gamma-glutamytranspeptidase (GGT). The red line designates the Bonferroni-adjusted genome-wide significance threshold at 6.5^{e-9} and the blue line the suggestive association threshold at 1.3^{e-7}.

FIG. 3: shows the biomarkers PRO-C1 (type I collagen pro-peptide), PRO-C3 (type Ill collagen pro-peptide), and PRO-C6 (type VI collagen pro-peptide) were measured in the CAF supernatant to reflect the fibrotic activity and collagen synthesis. The fold change between day 3 and day 9 was compared for the different doses of TGFβI treatment.

FIG. 4 shows the biomarker PRO-C3 (type Ill collagen pro-peptide) levels were measured in the Pancreatic fibroblast supernatant to reflect the fibrotic activity and collagen synthesis. The biomarker level was measured at day 12 for the different treatment doses of TGF-b, TGFβI, and EMI-domain.

FIG. 5 shows the effect of increasing levels of BigH3 (TGFβI) on the production of the markers of fibroblast activation in pancreatic fibroblasts. FIG. 5A shows Pro-C1 levels;

FIG. 5B shows Pro-C3 levels;

FIG. 5C shows Pro-C6 levels; and

FIG. 5D shows P3NP levels.

### EXAMPLES

The presently disclosed embodiments are described in the following Examples, which are set forth to aid in the understanding of the disclosure and should not be construed to limit in any way the scope of the disclosure as defined in the claims which follow thereafter. The following examples are put forth so as to provide those of ordinary skill in the art with a complete disclosure and description of how to make and use the described embodiments and are not intended to limit the scope of the present disclosure nor are they intended to represent that the experiments below are all or the only experiments performed. Efforts have been made to ensure accuracy with respect to numbers used (e.g. amounts, temperature, etc.) but some experimental errors and deviations should be accounted for.

Unless indicated otherwise, parts are parts by weight, molecular weight is weight average molecular weight, temperature is in degrees Centigrade, and pressure is at or near atmospheric.

### Materials and Methods

### Reagents

All reagents used for the experiments were standard chemicals from Merck (Whitehouse Station, NJ, USA) or Sigma Aldrich (St. Louis, MO, USA), unless otherwise stated. The synthetic immunogenic peptide Ovalbumin (OVA)-CGG-CPTGPQNYSP used for monoclonal antibody production was purchased from Chinese Peptide Company (Beijing, China).

### Generation of Pro-C3 Monoclonal Antibodies

Pro-C3 monoclonal antibody production was performed as previously reported [23](see also WO2014/170312A1). Briefly, the Pro-C3 neo-eptiope (the N-proteinase generated C-terminal neo-epitope of PIIINP described above) was selected as the target and the amino acid sequence 144‘-CPTGPQNYSP-’153 (SEQ. ID No: 4) in the α1 chain of PIIINP was used to generate an antibody specific for said target. The sequence was aligned for homology to rat and mouse and blasted for uniqueness among other human proteins using the NPS@: network protein sequence analysis with the Uniprot/Swiss-Prot database [24]. The sequence CPTGPQNYSP (SEQ. ID No: 4) was found unique for PIIINP.

Generation of monoclonal antibodies, clone characterization and antibody characterization were carried out as previously described [22]. The monoclonal antibody was found specific for the target sequence CPTGPQNYSP (SEQ. ID No: 4) and did not recognise or bind to an elongated (CPTGPQNYSPQ (SEQ. ID No: 5)) or non-sense peptide (GSPGKDGVRG (SEQ. ID No: 41)) [22].

Supernatant from antibody producing hybridoma cells was collected and the monoclonal antibody was purified using HiTrap protein-G-columns (GE healthcare Life Sciences, Little Chalfont, Buckinghamshire, UK) and labelled with the Lightning-Link™ Rapid Biotin Conjugation Kit (Type B) (Innova Biosciences) or Lightning-Link™ HRP Conjugation Kit (Innova Biosciences) according to the manufacturer's instructions.

The antibodies generated were sequenced and the CDRs determined.

The sequence of the chains are as follows (CDRs underlined and in bold, constant regions italics):

Heavy chain: Amino acid sequence (467 aa)
(SEQ. ID No: 42)
EVQLQQSGPEVLKPGASVKMSCKASGYTFINYVIHWLKQKAGQGPEWIGYMNPYNDVPKN
NAKFRGKARLTSDRSSTTAYMELNSLTSEDSAVYYCARGGFFGPLSYWGQGTLVTVSAAK
TTAPSVYPLAPVCGDTTGSSVTLGCLVKGYFPEPVTLTWNSGSLSSGVHTFPAVLQSDLYT
LSSSVTVTSSTWPSQSITCNVAHPASSTKVDKKIEPRGPTIKPCPPCKCPAPNLLGGPSVFIF
PPKIKDVLMISLSPIVTCVVVDVSEDDPDVQISWFVNNVEVHTAQTQTHREDYNSTLRVVSA
LPIQHQDWMSGKEFKCKVNNKDLPAPIERTISKPKGSVRAPQVYVLPPPEEEMTKKQVTLT
CMVTDFMPEDIYVEWTNNGKTELNYKNTEPVLDSDGSYFMYSKLRVEKKNWVERNSYSCS
VVHEGLHNHHTTKSFSRTPGK
Light chain: Amino acid sequence (238 aa)
(SEQ. ID No: 43)
DVLMTQTPLSLSVSLGDQASISCRSSQNIVYSNGDTYFEWYLQKPGQSPKLLIYKVSQRFS
GVPDRFSGSGSGTDFTLKISRVETEDLGVYYCFQGAHDPPAFGGGTKLELKRADAAPTVSI
FPPSSEQLTSGGASVVCFLNNFYPKDINVKWKIDGSERQNGVLNSWTDQDSKDSTYSMSS
TLTLTKDEYERHNSYTCEATHKTSTSPIVKSFNRNEC

### Pro-C3 Assay

The Pro-C3 assay (Pro-C3 competitive ELISA) was performed as previously reported [23](see also WO2014/170312A1 incorporated herein by reference). Briefly, the PRO-C3 competitive ELISA procedure was as follows: A 96-well streptavidin-coated ELISA plate from Roche, cat. 11940279, was coated with the biotinylated peptide Biotin-CGGPTGPQNYSP (SEQ. ID No: 4) dissolved in coater buffer (50 mM PBS-BTE+10% sorbitol, pH 7.4), incubated for 30 min at 20° C. in the dark and subsequently washed in washing buffer (20 mM Tris, 50 mM NaCl, pH 7.2). Thereafter 20 μl of peptide calibrator or sample were added to appropriate wells, followed by 100 μl of HRP-conjugated Pro-C3 monoclonal antibody (monoclonal antibody NB61N-62) dissolved in incubation buffer (50 mM PBS-BTB+10% Liquidll (Roche), pH 7.4) and the plate was incubated for 20 hours at 4° C. and washed. Finally, 100 μl tetramethylbenzinidine (TMB) (Kem-En-Tec cat.: 4380H) was added, the plate was incubated for 15 min at 20° C. in the dark and, to stop the reaction, 100 μl of stopping solution (1% H₂SO₄) was added and the plate was analysed in the ELISA reader at 450 nm with 650 nm as the reference (Molecular Devices, SpectraMax M, CA, USA). A calibration curve was plotted using a 4-parametric mathematical fit model.

### Antibody Development for PRO-C6

A monoclonal antibody specific for PRO-C6 was developed as described in WO 2016/156526 (Nordic Bioscience, incorporated herein by reference) using the last 10 amino acids of the type VI collagen α3 chain (i.e. the C-terminus sequence³¹⁶s‘KPGVISVMGT’³¹ m¹(SEQ ID No: 10)) as an immunogenic peptide. Briefly, 4-6-week-old Balb/C mice were immunized subcutaneously with 200 μl emulsified antigen with 60 μg of the immunogenic peptide.

Consecutive immunizations were performed at 2-week intervals in Freund's incomplete adjuvant, until stable sera titer levels were reached, and the mice were bled from the 2nd immunization on. At each bleeding, the serum titer was detected and the mouse with highest antiserum titer and the best native reactivity was selected for fusion. The selected mouse was rested for 1 month followed by intravenous boosting with 50μg of immunogenic peptide in 100 μl 0.9% sodium chloride solution 3 days before isolation of the spleen for cell fusion.

Mouse spleen cells were fused with SP2/0 myeloma fusion partner cells. The fusion cells were raised in 96-well plates and incubated in the CO₂-incubator. Here standard limited dilution was used to promote monoclonal growth. Cell lines specific to the selection peptide and without cross-reactivity to either elongated peptide (KPGVISVMGTA (SEQ ID No: 18), Chinese Peptide Company, China) or truncated peptide (KPGVISVMG (SEQ ID No: 19), American Peptide Company, USA) were selected and sub-cloned. At last the antibodies were purified using an IgG column.

The antibodies generated were sequenced and the CDRs determined.

The sequence of the chains are as follows (CDRs underlined and in bold):

Heavy Chain Sequence (mouse IgG1 isotype)
(SEQ ID No: 44)
EVQLQQSGPVMVKPGTSVKTSCKASGYTFTDFNMNWVKQSHGKSL
EWIGAINPHNGATSYNQKFSGKATLTVDKSSSTAYMELNSLTSDD
SAVYYCARWGNGKNSWGQGTTLTVSSAKTTPPSVYPLAPGSAAQT
NSMVTLGCLVKGYFPEPVTVTWNSGSLSSGVHTFPAVLQSDLYTL
SSSVTVPSSTWPSETVTCNVAHPASSTKVDKKIVPRDCGCKPCIC
TVPEVSSVFIFPPKPKDVLTITLTPKVTCVVVDISKDDPEVQFSW
FVDDVEVHTAQTQPREEQFNSTFRSVSELPIMHQDWLNGKEFKCR
VNSAAFPAPIEKTISKTKGRPKAPQVYTIPPPKEQMAKDKVSLTC
MITDFFPEDITVEWQWNGQPAENYKNTQPIMDTDGSYFVYSKLNV
QKSNWEAGNTFTCSVLHEGLHNHHTEKSLSHSPGK
CDR-H1:
(SEQ ID No: 23)
DFNMN
CDR-H2:
(SEQ ID No: 24)
AINPHNGATSYNQKFSG
CDR-H3:
(SEQ ID No: 25)
WGNGKNS
Light Chain Sequence (mouse Kappa isotype)
(SEQ ID No: 45)
DVVMTQTPLSLPVNLGDQASISCRSSQRIVHSNGITFLEWYLQKP
GQSPKLLIYRVSNRFSGVPDRFSGSGSGTDFTLKISRVEAEDLGL
YYCFQGSHVPLTFGAGTRLELKRADAAPTVSIFPPSSEQLTSGGA
SVVCFLNNFYPKDINVKWKIDGSERQNGVLNSWTDQDSKDSTYSM
SSTLTLTKDEYERHNSYTCEATHKTSTSPIVKSFNRNEC
CDR-L1:
(SEQ ID No: 20)
RSSQRIVHSNGITFLE
CDR-L2:
(SEQ ID No: 21)
RVSNRFS
CDR-L3:
(SEQ ID No: 22)
FQGSHVPLT

### Immunoassay for PRO-C6.

PRO-C6 was measured using an enzyme-linked immunosorbent assay (ELISA) developed at Nordic Bioscience, as described in WO2016/156526 (incorporated herein by reference), and as also detailed in other publications. Briefly, these procedures were as follows: ELISA-plates used for the assay development were Streptavidin-coated from Roche (cat.: 11940279). All ELISA plates were analyzed with the ELISA reader from Molecular Devices, SpectraMax M, (CA, USA). We labeled the selected monoclonal antibody with horseradish peroxidase (HRP) using the Lightning link HRP labeling kit according to the instructions of the manufacturer (Innovabioscience, Babraham, Cambridge, UK). A 96-well streptavidin plate was coated with biotinylated synthetic peptide biotin-KPGVISVMGT (SEQ ID No: 38) (Chinese Peptide Company, China) dissolved in coating buffer (40 mM Na₂HPO₄, 7 mM KH₂PO₄, 137 mM NaCl, 2.7 mM KCl, 0.1% Tween 20, 1% BSA, pH 7.4) and incubated 30 minutes at 20° C. 20 μL of standard peptide or samples diluted in incubation buffer (40 mM Na₂HPO₄, 7 mM KH₂PO₄, 137 mM NaCl, 2.7 mM KCl, 0.1% Tween 20, 1% BSA, 5% Liquid II, pH 7.4) were added to appropriate wells, followed by 100 μL of HRP conjugated monoclonal antibody 10A3, and incubated 21 hour at 4° C. Finally, 100 μL tetramethylbenzinidine (TMB) (Kem-En-Tec cat.4380H) was added and the plate was incubated 15 minutes at 20° C. in the dark. All the above incubation steps included shaking at 300 rpm. After each incubation step the plate was washed five times in washing buffer (20 mM Tris, 50 mM NaCl). The TMB reaction was stopped by adding 100 μL of stopping solution (1% H₂SO₄) and measured at 450 nm with 650 nm as the reference.

### Immunoassay for Pro-C1

Immunoassay for detecting Pro-C1 was obtained from Nordic Bioscience (cat #2800A0E51), and carried out in accordance with the manufacturer's instructions.

### Immunoassay for P3NP

Immunoassay for detecting P3NP was obtained from Nordic Bioscience (cat #1090BD01), and carried out in accordance with the manufacturer's instructions.

### Study Design

The Prospective Epidemiologic Risk Factor (PERF) study, was a follow-up study of Danish post-menopausal women aiming at identifying risk factors associated with age-related diseases [25]. Participants in PERF have previously participated in clinical randomized placebo-controlled studies or were screened without being randomized for previous studies at the Center for Clinical and Basic Research (CCBR). The study participant inclusion process was performed in the same ways as in [26]. A total of 4968 subjects with PRO-C3 measurements, serum biochemistry measurements, medical history and genotypes have been included.

### Standard Protocol Approvals, Registrations, and Patient Consents

The study was carried out in accordance with the International Conference on Harmonization—Guideline for Good Clinical Practice (ICH-GCP) and the study protocol was approved by the local ethics committees. All participants signed an informed consent allowing future analysis to be performed.

### Baseline Measurements and Data Collection

At baseline the participants completed an interview with a doctor or a nurse covering questions related to physical health, demographics, lifestyle and medical history. Fasting serum and DNA samples were collected from subjects who gave written consent for this specific analysis.

PRO-C3 levels were measured blinded in serum by enzyme-linked immunosorbent assay (ELISA) in a CAP-certified laboratory as previously described. Lymphocyte and neutrophil counts were determined using an automated blood cell analyser (Sysmex).

Complete hospital disease history of the subjects was obtained for the period (1974-2014) by linking each individual's unique personal identification number (CPR Number) with the Danish patient registries on 31-12-2014 corresponding to end of study. Participants of the study were anonymized, and CPR numbers were not made available at any point of the study. Patient registry information was available for 5602 participants.

Genotyping was performed using a custom-made Illumina Global Screening Array (693143 probes) in collaboration with deCODE Genetics, Iceland. SNP imputation was carried out using the Michigan Imputation Server [27], using a HRC r1.1.2016, EUR reference panel. Phasing was performed with Shapelt2 and the imputation with Minimac3. Positions are reported as in the GRCh37 reference.

Disease phenotype definition Eighteen disease phenotypes have been defined as all-time incidence of an event based on data available from multiple sources: biochemical marker levels, physiological measurements, all-time incidence hospital records, death registry, questionnaires taken during baseline and follow-up visits. The detailed list of included phenotypes and their inclusion criteria is provided in supplementary table 2.

### Genotype Data Pre-Processing

Standard probe-level filtering has been performed using a minimum probe call rate of 97%, a minor allele frequency greater or equal to 1% and a Hardy-Weinberg Equilibrium p-value cut-off greater or equal to 1e-6. No filtering on multi-allelic SNPs has been performed.

To address possible cryptic relatedness between subjects, we calculated an identity-by-descent (IBD) coefficient using Plink[18]--genome function. Inbreeding coefficient (IBC) was calculated using the plink--ibc function. Subjects were removed, on a one side of a pair basis, using a minimum P/_HAT cut-off value of 0.1875 and a cut-off of less than −0.1 or greater than 0.1 was applied to the Fhat2 coefficient.

### Principal Component Analysis

Population-based genetic variation in the dataset was captured using EIGENSTRAT Smartpca 7.2.0 [28,29] to perform an iterative principal component analysis (PCA) of the study population with available genotypes on the non-imputed filtered variants using the default parameters.

### Robust Elastic Net Covariable Selection

A robust and optimized variable selection procedure was used to include the variables in the GWAS. A 50 times randomized, 5-fold cross-validated elastic net was performed to select variables associated with log 2 levels of PRO-C3. Visualization of the distribution of coefficient estimates was done using standard geom_boxplots.

### Linear Regression

Linear additive regression was performed on the GWAS study population (n=4968) to identify genetic associations with log 2-transformed serum PRO-C3 levels using plink v1.90p [30](Chang et al. 2015) adjusted for baseline age and the three leading principal components. Conservative significance thresholds based on the number of screened variants were defined as equal to 6.5e-9 i.e. 0.05/N, and 1.3^{e-7}, i.e. 1/N, N=7672338, for the genome-wide and suggestive values respectively. The Manhattan-plot visualizations were made using the R package qqman [31].

### Phenotype-Association Analysis

Phenotype-association analysis with log 2(PRO-C3) levels was performed using a logistic regression model, corrected for baseline age, BMI. Statistical significance was adjusted using a Benjamini-Hochberg method. P-values and betas of log 2(PRO-C3) association with disease were visualized as a Forest plot using the forestplot R library.

### Correlation Analysis

To assess the correlation between PRO-C3 and different liver related variables and disease scores, and Pearson's correlation test has been performed. Linear values of biochemical concentration were considered.

To assess the correlation between the genetic components associated with PRO-C3 and different liver related variables and disease scores, a genetic correlation analysis [32] was performed. GWAS analyses were run as described above for each variable, using a standard model correcting for age at baseline and the three leading principal components were used to perform these GWAS. Log2-transformed values of biochemical measurements were considered in the GWAS. We used LD scores estimated from the PERF genotyping array data.

### Pathway Enrichment Analysis

Pathway enrichment analysis was performed using VEGAS2 [33] and PARIS 2.4 [34] programs using default parameters. VEGAS2 analysis used the Biosystems gene/pathway annotation file provided by the software website. The LOKI knowledge base used by PARIS2.4 was compiled by running the script provided by the program in February 2020. Overlapping significant associations to REACTOME pathways with p<0.05 to both frameworks were reported.

### Data Availability

The original data of the Prospective Epidemiological Risk Factor study and the linkage data from various health registries are currently stored at Nordic Bioscience. Access to this database may be granted, on condition that researchers have appropriate ethical permission and sign the appropriate Material Transfer Agreement form.

### Example N

A total of 4968 subjects from the PERF cohort were included in the study with both PRO-C3, serum biomarker measurements at baseline, hospital admissions from the Danish Patient Registry and genotypes. Inclusion criteria are described in the methods section and the previous study [26]. Table 1 summarizes the baseline characteristics of the subjects.

TABLE 1
Baseline characteristics of subjects included in the study

| Variable | N | Median(Q1-Q3) or n | |
|---|---|---|---|
| Baseline age (years) | 4968 | 70.0 | (10.0) |
| Education | 4964 | | |
| Primary_school | | 3549 | |
| High_school | | 1061 | |
| University | | 354 | |
| BMI | 4968 | 25.7 | (5.4) |
| Systolic bp (mmHg) | 4966 | 148.0 | (33.0) |
| Diastolic bp (mmHg) | 4968 | 81.0 | (16.0) |
| Serum PRO-C3 (ng/mL) | 4968 | 9.0 | (3.6) |
| ADAPT | 4968 | 5.0 | (1.1) |
| FIB4 | 4968 | 1.4 | (0.6) |
| APRI | 4968 | 0.2 | (0.1) |
| NFS | 4968 | 0.9 | (1.2) |
| Smoking | 4968 | | |
| Never | | 2349 | |
| Previous smoker | | 1515 | |
| Current smoker | | 1104 | |
| Alcohol | 4948 | | |
| <7 units per week | | 3326 | |
| >7 units per week | | 1622 | |
| Exercise | 4967 | | |
| once per week | | 1051 | |
| twice per week | | 651 | |
| >twice per week | | 1756 | |
| Never | | 1509 | |
| Serum ALB (mmol/L) | 4968 | 0.6 | (0.1) |
| Serum ALT (unit/L) | 4968 | 24.0 | (10.0) |
| Serum AP (unit/L) | 4968 | 166.0 | (51.0) |
| Serum GGT (unit/L) | 4968 | 29.0 | (18.0) |
| Serum AST (unit/L) | 4968 | 24.0 | (6.0) |
| Serum CHOL (mmol/L) | 4968 | 6.3 | (1.4) |
| Blood WBC | 4968 | 5.6 | (1.9) |

Disease Incidence Associated with Elevated PRO-C3 Levels

To elucidate the disease profile of PERF subjects in relation to their PRO-C3 levels, a logistic regression analysis was performed, adjusted for the baseline characteristics of the subjects (age and BMI). Log levels of PRO-C3 levels are strongly associated with increased incidence of chronic liver disease (OR=1 0.74, 95% CI [1.45-2.09], adjusted p=1.09e-7, and nominally associated with chronic kidney disease (OR=1.26, 95% CI [1.10-1.24], adjusted p=6.04e-3) and rheumatic disease (OR=1.35, 95% CI [1.13-1.63], adjusted p=6.69^{e-3}).

The logistic regression was performed again using PRO-C3 levels binned into quartiles, shows that the association resides more specifically within Q4 PRO-C3 for liver and rheumatic disease and Q3-Q4 for chronic kidney disease. It is noted that the fit accuracy is modest, albeit the strong p-values: AUC=0.60 for Liver and Rheumatic disease, 0.70 for kidney disease.

### Genome-Wide Association Study

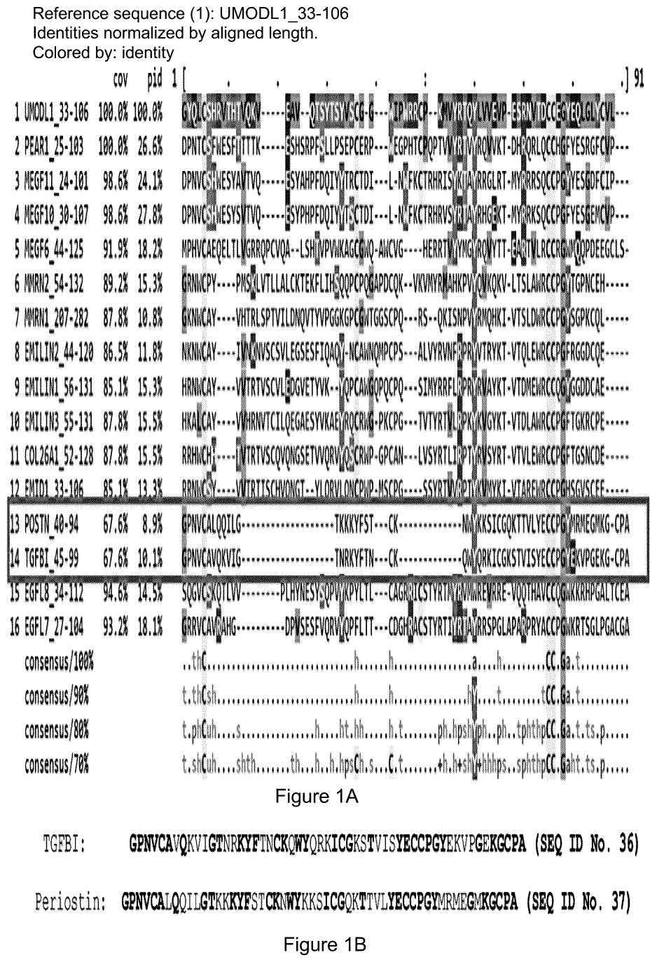

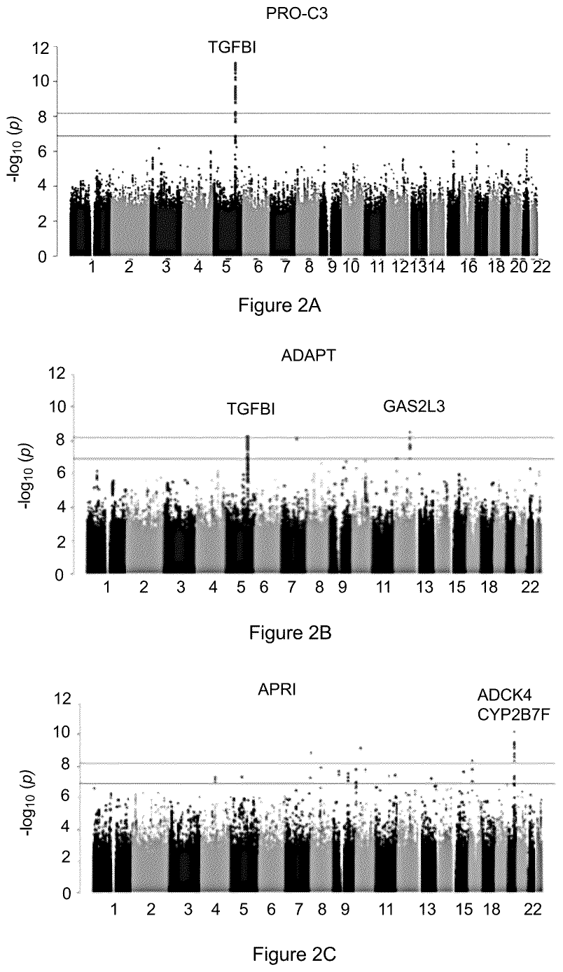

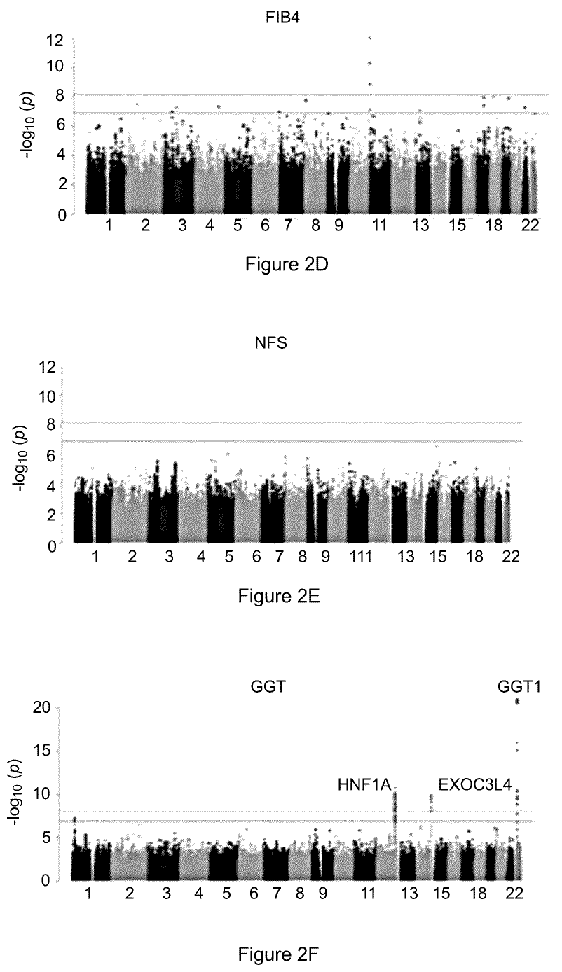

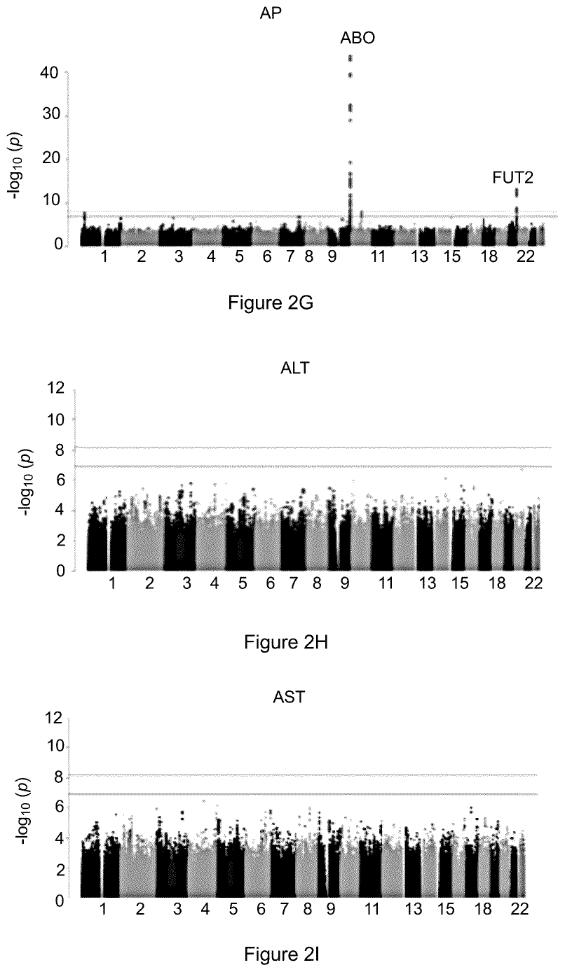

A GWAS analysis on log 2-transformed PRO-C3 levels at baseline was performed using an optimized variable selection procedure consisting of a cross-validated elastic net. The model was adjusted for log 2-transformed values of the following serum and blood biochemical measurements at baseline: alkaline phosphatase (AP), aspartate aminotransferase (AST), sodium (NA) and middle cell hemoglobin concentration (MCHC) as well as PRO-C3 measurement batch number. The results summarized as a Manhattan plot are shown in FIG. 2. Log2(PRO-C3) levels are associated with TGFBI/VNTRNA2-1 locus on Chromosome 5 with lead SNP rs2073511 (FIG. 2, effect size 0.070, 95% CI [0.050-0.090]log 2 ng/mL, p-value=8.42e-12). Significant variants in the locus are across the genic area of the TGFBI gene and in strong linkage.

The study population was stratified according to their log 2-transformed PRO-C3 quartiles and made a contingency table of occurrences of rs2073511 alleles within these groups. The results suggest that the TGFBI lead variant is overrepresented in subjects belonging to the top PRO-C3 quartile (Chi-square p-value: 1.177e-07).

Correlation with Other Liver Relevant Biomarkers and Scores

To better elucidate the relationship between PRO-C3 and chronic liver disease, the correlation between PRO-C3 levels and standard biochemical markers that have been linked with liver disease were investigated, as well as liver associated disease scores. For this, Pearson's correlation analysis was performed to further decipher the potential confounding effects on PRO-C3 biomarker.

The Pearson's correlation analysis shows that PRO-C3 levels correlate moderately with serum levels of alanine aminotransferase (ALT), alkaline phosphatase (AP), gamma-glutamytranspeptidase (GGT), aspartate aminotransferase (AST) (Pearson's correlation range [0.13-0.19]). With regards to disease scores, PRO-C3 is tagged to ADAPT score (Pearson's correlation 0.83), which is expected since it is a component of the score calculation.

GWAS was performed on the liver biomarkers AP, ALT, AST, GGT and the scores ADAPT, APRI, FIB4, NFS, adjusted for age at baseline and 3 leading principal components (FIG. 2).

While looking at the genetic correlation [32] of the variants associated with each of these different markers and scores, the genetic landscape associated with PRO-C3 is not correlated to any of those of other biochemical markers or disease scores, except for ADAPT and the correlation pattern differs from that calculated based on Pearson's correlation of levels and scores. These results suggest that the association between TGFBI variants association is specific to PRO-C3.

### Example 2

### In Vitro Study

By applying the “Scar-in-α-jar” (SiaJ) model approach previously described (35)(36), pancreatic CAFs were grown to confluency and seeded in a 96 well plate with a cell density of 10,000 cells/well in 200 μl 10% FBS Dulbecco's modified eagle medium (DMEM)+GlutaMax per well. The day after seeding the supernatant was removed and the cells received TGFBI treatment in the following concentrations: 0 (vehicle control), 1 ng/ml, 10 ng/ml, and 100 ng/ml in 200 μl 0.4% FBS DMEM per well in quadruplicates. 3 and 6 days after first treatment, the supernatant was removed and stored at −20° and new TGFBI treatment in 0.4% FBS DMEM was added to the cells. 9 days after the first treatment the supernatant was removed and stored, and the experiment terminated. The biomarkers PRO-C1 (type I collagen pro-peptide), PRO-C3 (type Ill collagen pro-peptide), and PRO-C6 (type VI collagen pro-peptide) were measured in the supernatant to reflect the fibrotic activity and collagen synthesis from the different days of the SIAJ setup. The fold change between day 3 and day 9 was compared for the different doses of TGFβI treatment.

In an additional setup normal pancreatic fibroblast were grown and seeded (similar to the CAFs) followed by treatment with TGF-3 (0.08 nM), TGFBI (5 nM and 100 nM) and with the 56 amino acid EMI-domain from Periostin in the following concentrations: 0 (vehicle control), 1 nM, 10 nM, and 100 nM in 200 μl 0.4% FBS DMEM per well in quadruplicates. 3, 6, and 9 days after first treatment the supernatant was removed and stored at −20° C. and new treatment in 0.4% DMEM was added to the cells. The experiment was terminated after supernatant removal 12 days after first treatment. PRO-C3 was measured in supernatant and the levels from the different treatment doses were compared. The Native human quiescent pancreatic fibroblasts and pancreatic CAFs were purchased from Neuromics (cat #SCOOA05 and cat #CAF08, respectively, US).

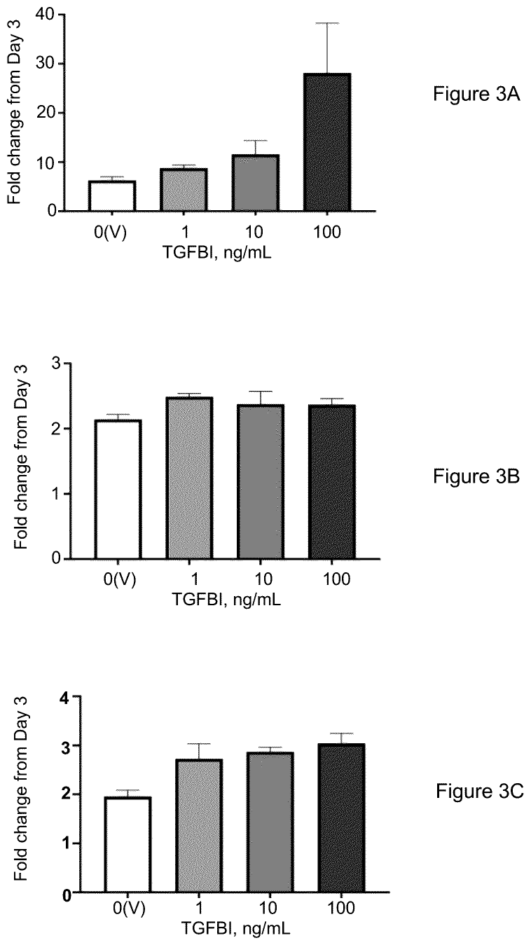

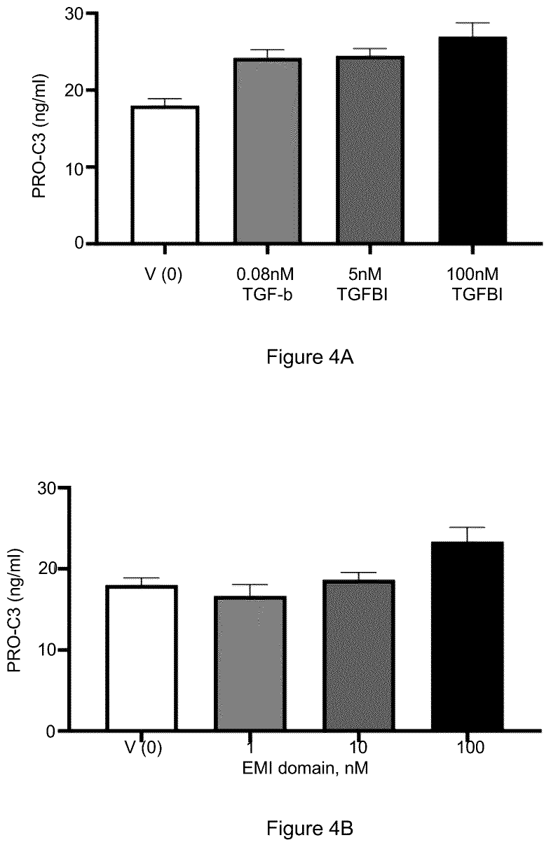

Results: As shown in FIG. 3, most notably a clear dose dependent increase in PRO-C3 levels was seen in with CAFs treated with TGFBI. In CAFs treated with 100 ng/mL TGBI the increase from day 3 to day 9 in PRO-C3 levels was approximately 28-fold compared to approximately 6-fold in untreated CAFs, corresponding to more than 450% increase in PRO-C3 levels in CAFs treated with 100 ng/ml TGFBI compared to untreated CAFs. A smaller (50%) dose-dependent increase was also observed for PRO-C6 levels in the supernatant from TGFβI treated CAFs compared to the untreated CAFs. There was no change in the PRO-C1 levels between TGFβI treated CAFs and untreated CAFs. As shown in FIG. 4a 5 nM TGFBI induced PRO-C3 levels to the same extent as TGF-β in normal pancreatic fibroblast (35% and 34% increase from vehicle control respectively) and high levels of TGFBI (100 nM) increased the induction even more (50% increase from vehicle control) in normal pancreatic fibroblasts. Interestingly 100 nM of the EMI-domain peptide also resulted in an induction of PRO-C3 levels (30% increase from vehicle control) in normal pancreatic fibroblasts.

Conclusions: TGFβI stimulation of pancreatic normal fibroblasts and CAFs led to a dose dependent increase in specific collagen synthesis, in particular synthesis of type Ill collagen (PRO-C3). These results suggest a direct association between TGFβI, CAF activity, collagen synthesis and PRO-C3 levels, and support TGFβI as a novel anti-fibrotic/anti-cancer drug target. In addition, stimulation with an EMI-domain peptide also led to increased PRO-C3 levels, altogether indicating a potential role of the EMI-domain of TGFβI and thereby potentially specifying the TGFβI targeting for anti-fibrotic/anti-cancer drug targeting.

### Example 3 Fibroblasts Cultures (Scar in a Jar)

Pancreatic fibroblasts (PFs) were cultured in VitroPlus Ill, low serum (Neuromics cat #PCOOB1, Edina, MN, USA) in rat tail type I collagen (cat #P8188, Innoprot, Derio, Bizkaia, Spain) coated culture flask (5μg/cm2) at 37° C., 5% CO2. When the cells reached 80% confluency, they were seeded in 96-well plates with a cell density of 15,000 cells per well in Gibco Dulbecco's modified eagle medium+GlutaMAX (DMEM) (cat #31966047, Thermo Fisher Scientific, Waltham, MA, USA) supplemented with 10% fetal bovine serum (FBS) (cat #F7524, Sigma Aldrich, St. Louis, MO, USA) and 1% penicillin/streptavidin (P/S) (cat #P4333, Sigma Aldrich, USA). Prior to seeding the cells, a high binding 96-well plate (cat #3361, Corning, New York, USA) was coated with either PBS, 2% BSA, or different concentrations of BigH3 100 μl/well were used (50 nm, 100nnm, 200 nm and 400 nm), and the plate were incubated for 1 h at 37° C. After coating, the plate was washed once with 200 μl/well PBS and then blocked with 200 μl/well 2% BSA in PBS for 20 min at 37° C. After 24 hours (day 0), the media was changed to Ficoll media (DMEM with 0.4% FBS, 1% P/S, 56.25 mg/mL 70 kDa Ficoll (cat #17-0310-50 GE healthcare, Chicago, IL, USA), 37.5 mg/mL 400 kDa Ficoll (17-0300-50, GE healthcare, Chicago, IL, USA), and 0.05 mg/mL L-ascorbic acid (cat #A9256, Sigma Aaldrich, St. Louis, MO, USA)). Every third day the supernatants were removed and frozen, and at termination (day 12).

### Assessment of Soluble Biomarkers

BigH3 (Abcam, cat #ab220651) and PRO-C3 (Nordic Bioscience, cat #1700AF06) were measured blinded in serum by enzyme-linked immunosorbent assays (ELISAs) or Chemiluminescent Immunosorbent Assays (CLIAs) in accordance with the manufacturer's instructions. Similarly, PRO-C1 (Nordic Bioscience, cat #2800A0E51), PRO-C6 (Nordic Bioscience, cat #4000AF02) and P3NP (Nordic Bioscience, cat #1090BD01) were measured blinded in serum by enzyme-linked immunosorbent assays (ELISAs) in accordance with the manufacturer's instructions.

### BIGH3 is an Inducer of PRO-C3 (and Other Collagens) in Pancreatic Fibroblasts

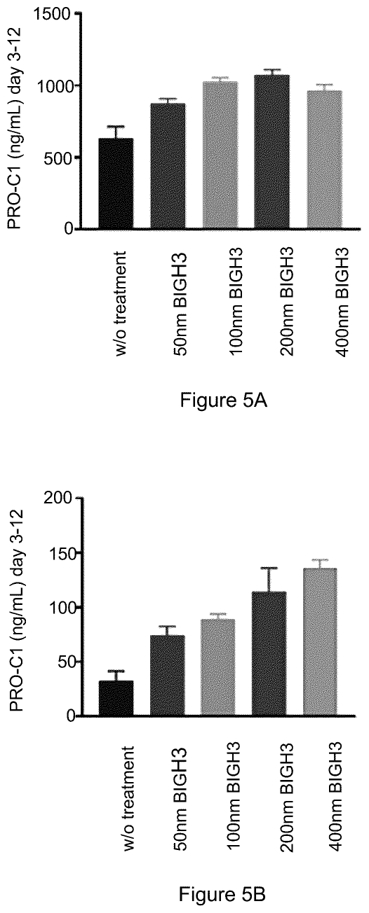

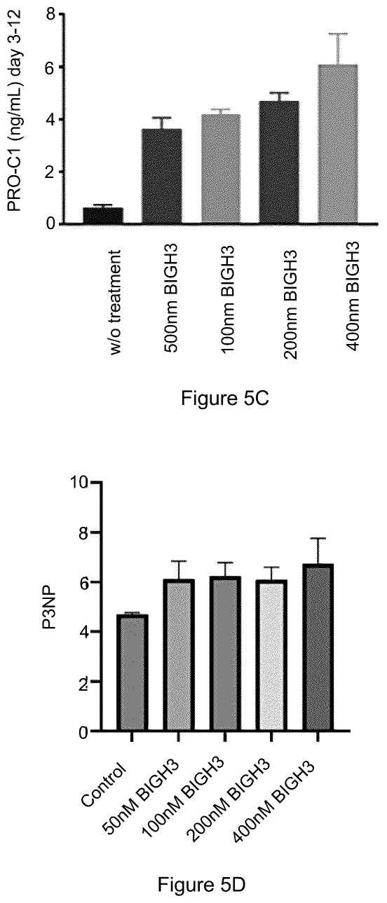

After establishing a correlation between BigH3 and PRO-C3, experiments were carried out to determine whether BIGH3 was also an inducer of PRO-C3 in pancreas fibroblasts cultured in vitro in the scar-in-α-jar model. As shown in FIG. 5, incubating pancreas fibroblasts in the presence of increasing doses (50 nM to 400 nM) of BIGH3 resulted in dose-dependent increases in PRO-C3. Moreover, a similar phenomenon was seen for the type I collagen synthesis biomarker PRO-C1 and the type VI collagen synthesis biomarker PRO-C6, as well as the classical fibrosis indicator P3NP (FIG. 5). Taken together this indicates that BigH3 is an inducer of PRO-C3 and other collagens in pancreatic fibroblasts suggesting that BigH3 may be a driver of fibroblast activation to produce more collagen and fibrosis. Importantly, the induction of collagen type Ill synthesis was measured using PRO-C3, and thereby links directly to the initial finding of a relationship between the BIGH3 SNPs and PRO-C3 levels in circulation. Furthermore, it also suggests that BIGH3 may be an important therapeutic target for tumor fibrosis and cancer associated fibroblast modulation in cancer patients with high PRO-C3.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used to mean ‘including or consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

### REFERENCES

1. Jenkins R G, et al. Longitudinal change in collagen degradation biomarkers in idiopathic pulmonary fibrosis: an analysis from the prospective, multicentre PROFILE study. Lancet Respir Med. 2015a;3:462-72.
2. Organ L A, et al. Biomarkers of collagen synthesis predict progression in the PROFILE idiopathic pulmonary fibrosis cohort. Respir Res.2019; 20:148.
3. Kristensen J H, et al. Levels of circulating MMP-7 degraded elastin are elevated in pulmonary disorders. Clin Biochem. 2015; 48:1083-8.
4. Leeming D J, et al. Serological investigation of the collagen degradation profile of patients with chronic obstructive pulmonary disease or idiopathic pulmonary fibrosis. Biomark Insights. 2012; 7:119-26.
5. Karsdal M A, et al. The good and the bad collagens of fibrosis-their role in signalling and organ function. Adv Drug Deliv Rev.2017; 121:43-56.
6. Bao X, Zeng Y, Wei S, Wang G, Liu C, Sun Y, Chen Q, and Li H. Developmental changes of Col3a1 mRNA expression in muscle and their association with intramuscular collagen in pigs. J Genet Genomics 2007; 34(3): 223-228.
7. Jensen L T and Host N B. Collagen: scaffold for repair or execution. Cardiovasc Res 1997; 33(3): 535-539.
8. Niemela O, Risteli L, Parkkinen J, and Risteli J. Purification and characterization of the N-terminal propeptide of human type Ill procollagen. Biochem J 1985; 232(1): 145-150.
9. Wang W M, Ge G, Lim N H, Nagase H, and Greenspan D S. TIMP-3 inhibits the procollagen N-proteinase ADAMTS-2. Biochem J 2006; 398(3): 515-519.
10. Goehrig D et al. Stromal protein pig-h3 reprogrammes tumour microenvironment in pancreatic cancer [Internet]. Gut 2019; 68(4):693-707.
11. Sato T et al. Identification and characterization of transforming growth factor beta-induced in circulating tumor cell subline from pancreatic cancer cell line [Internet]. Cancer Sci. 2018; 109(11):3623-3633.
12. Nacu N et al. Macrophages produce TGF-beta-induced (beta-ig-h3) following ingestion of apoptotic cells and regulate MMP14 levels and collagen turnover in fibroblasts [Internet]. J. Immunol. 2008; 180(7):5036-5044.
13. Tachmazidou I, Hatzikotoulas K, Southam L, et al. Identification of new therapeutic targets for osteoarthritis through genome-wide analyses of UK Biobank data. Nat Genet 2019; 51:230-6. doi:10.1038/s41588-018-0327-1
14. Nielsen M J, Veidal S S, Karsdal M A, et al. Plasma Pro-C3 (N-terminal type Ill collagen propeptide) predicts fibrosis progression in patients with chronic hepatitis C. Liver Int 2015; 35:429-37.
15. Daniels S J, Leeming D J, Eslam M, et al. ADAPT: An Algorithm Incorporating PRO-C3 Accurately Identifies Patients With NAFLD and Advanced Fibrosis. Hepatology 2019;69. doi:10.1002/hep.30163
16. Nielsen M J, Nedergaard A F, Sun S, et al. The neo-epitope specific PRO-C3 ELISA measures true formation of type Ill collagen associated with liver and muscle parameters. Am J Transl Res 2013; 5:303-15.
17. Hirschfield G M, Chazouilleres O, Drenth J P, et al. Effect of NGM282, a FGF19 analogue, in Primary Sclerosing Cholangitis: a Multicentre, Randomized, Double-Blind, Placebo-Controlled Phase 2 Trial. J Hepatol 2018.
18. Bril F, Leeming D J, Karsdal M A, et al. Use of plasma fragments of propeptides of Type Ill, V, and VI procollagen for the detection of liver fibrosis in type 2 diabetes. Diabetes Care 2019;42. doi:10.2337/dc18-2578
19. Kai Ruan, et al. The multifaceted role of periostin in tumorigenesis Cell Mol Life Sci. 2009 Jul;66(14):2219-30 doi: 10.1007/s00018-009-0013-7
20. Deane F Mosher et al. Periostin and TGF-p-induced protein: Two peas in a pod?Crit Rev Biochem Mol Biol. 2015; 50(5):427-39. doi: 10.3109/10409238.2015.1069791.
21. Stefano Marastoni, et al. EMILIN2 down-modulates the Wnt signalling pathway and suppresses breast cancer cell growth and migration. J Pathol. 2014 March;232(4):391-404. doi: 10.1002/path.4316.
22. Nielsen M J, Nedergaard A F, Sun S, et al. The neo-epitope specific PRO-C3 ELISA measures true formation of type Ill collagen associated with liver and muscle parameters. Am J Transl Res. 2013; 5(3):303-315.
24. Combet C, Blanchet C, Geourjon C, Deleage G. NPS@: network protein sequence analysis. Trends Biochem Sci. 2000; 25(3):147-150.
25. Neergaard J S, Dragsbmk K, Kehlet S N, et al. Cohort Profile: The Prospective Epidemiological Risk Factor (PERF) Study. Int J Epidemiol 2016;1-10. doi:10.1093/ije/dyw251
26. Tang M-H E, Blair J P M, Bager C L, et al. Matrix metalloproteinase-degraded type I collagen is associated with APOE/TOMM40 variants and preclinical dementia. Neurol Genet 2020;6: e508. doi:10.1212/NXG.0000000000000508
27. Das S, Forer L, Sch6nherr S, et al. Next-generation genotype imputation service and methods HHS Public Access Author manuscript. Nat Genet 2016; 48:1284-7. doi:10.1038/ng.3656
28. Price A L, Patterson N J, Plenge R M, et al. Principal components analysis corrects for stratification in genome-wide association studies. Nat Genet 2006; 38:904-9. doi:10.1038/ng1847
29. Patterson N, Price A L, Reich D. Population structure and eigenanalysis. PLoS Genet 2006; 2:190. doi:10.1371/journal.pgen.0020190
30. Chang C C, Chow C C, Tellier L C, et al. Second-generation PLINK: rising to the challenge of larger and richer datasets. 2015; 4:7. doi:10.1186/s13742-015-0047-8
31. Turner S D. qqman: an R package for visualizing GWAS results using Q-Q and manhattan plots. bioRxiv 2014;005165. doi:10.1101/005165
32. Bulik-Sullivan B, Finucane H K, Anttila V, et al. An atlas of genetic correlations across human diseases and traits. Nat Genet 2015; 47:1236-41. doi:10.1038/ng.3406
33. Mishra A, Macgregor S. VEGAS2: Software for more flexible gene-based testing. Twin Res Hum Genet 2015; 18:86-91. doi:10.1017/thg.2014.79
34. Butkiewicz M, Cooke Bailey J N, Frase A, et al. Pathway analysis by randomization incorporating structure-PARIS: an update. Bioinformatics 2016; 32:2361-3. doi:10.1093/bioinformatics/btw130
35. Ronnow S R et al. Prolonged Scar-in-α-Jar: an in vitro screening tool for anti-fibrotic therapies using biomarkers of extracellular matrix synthesis. Respir. Res. 2020;21(1):108.
36. Nissen N I, Karsdal M, Willumsen N. Abstract 3958: Establishment of an in vitro model to study cancer associated fibroblasts and their direct effect on desmoplasia [Internet]. In: Cancer Research. American Association for Cancer Research (AACR); 2020:3958-3958

1. A method for identifying a patient who is more likely to respond to anti-TGFβI therapy, said method using an immunoassay for detecting in a patient sample at least one marker of fibroblast activation, said method comprising:
contacting said patient sample with a monoclonal antibody, wherein said monoclonal antibody is specifically reactive with an epitope of the marker of fibroblast activation;
determining the amount of binding of said antibody; and
correlating said amount of binding with values associated with normal healthy subjects, and/or with values associated with known responders to anti-TGFβI therapy, and/or with a predetermined cut-off value.

2. The method of claim 1, wherein the marker of fibroblast activation is selected from PRO-C3 (PIIINP), PRO-C6, PRO-C1 and/or P3NP.

3. The method of claim 1, wherein said method comprises:
contacting said patient sample with a monoclonal antibody, wherein said monoclonal antibody specifically binds an epitope of PIIINP;
determining the amount of binding of said antibody; and
correlating said amount of binding with values associated with normal healthy subjects, and/or with values associated with known responders to anti-TGFβI therapy, and/or with a predetermined cut-off value.

4. The method of claim 3, wherein the epitope of PIIINP with which the monoclonal antibody specifically binds is a C-terminal neo-epitope of PIIINP comprised in a C-terminal amino acid sequence CPTGXQNYSP-COOH wherein X is Gly or Pro (SEQ ID. No: 1).

5. The method of claim 4, wherein the monoclonal antibody does not specifically recognise or bind to an elongated version of said C-terminal amino acid sequence which is CPTGXQNYSPQ-COOH (SEQ ID. No: 5) and/or a truncated version of said C-terminal amino acid sequence which is CPTGXQNYS-COOH (SEQ ID. No: 6).

6. The method of claim 3, wherein the monoclonal antibody is raised against a synthetic peptide comprising or consisting of the C-terminal amino acid sequence CPTGXQNYSP-COOH wherein X is Gly or Pro (SEQ ID. No: 1).

7. The method of claim 1 wherein said method comprises:
contacting said patient sample with a monoclonal antibody, wherein said monoclonal antibody specifically binds a C-terminal epitope of the C5 domain of the α3 chain of type VI collagen;
determining the amount of binding of said antibody; and
correlating said amount of binding with values associated with normal healthy subjects, and/or with values associated with known responders to anti-TGFβI therapy, and/or with a predetermined cut-off value.

8. The method of claim 7, wherein the monoclonal antibody which specifically binds the C-terminal epitope of the C5 domain of the α3 chain of type VI collagen specifically binds to the C-terminus amino acid sequence KPGVISVMGT (SEQ ID No: 17).

9. The method of claim 7, wherein the monoclonal antibody does not specifically recognise or bind to an elongated version of said C-terminal amino acid sequence which is KPGVISVMGTA (SEQ ID No: 18) and/or a truncated version of said C-terminal amino acid sequence which is KPGVISVMG (SEQ ID No: 19).

10. The method of claim 7, wherein the monoclonal antibody is raised against a synthetic peptide comprising or consisting of the C-terminal amino acid sequence KPGVISVMGT (SEQ ID No: 17).

11. The method of claim 1 wherein said method comprises:
contacting said patient sample with a monoclonal antibody, wherein said monoclonal antibody specifically binds an N-terminal epitope of the pro-peptide of collagen type I;
determining the amount of binding of said antibody; and
correlating said amount of binding with values associated with normal healthy subjects, and/or with values associated with known responders to anti-TGFβI therapy, and/or with a predetermined cut-off value.

12. The method of claim 11, wherein the monoclonal antibody which specifically binds the N-terminal epitope of the pro-peptide of collagen type I specifically binds to the N-terminal amino acid sequence PDGSESPTDQETTGV (SEQ ID No: 3).

13. The method of claim 11, wherein the monoclonal antibody does not specifically recognise or bind to an elongated version of said C-terminal amino acid sequence which is PDGSESPTDQETTGVE (SEQ ID No: 15) and/or a truncated version of said C-terminal amino acid sequence which is DGSESPTDQETTGV (SEQ ID No: 17).

14. The method of claim 11, wherein the monoclonal antibody is raised against a synthetic peptide comprising or consisting of the N-terminus amino acid sequence PDGSESPTDQETTGV (SEQ ID No: 3).

15. The method of claim 1, wherein said method comprises:
contacting said patient sample with a monoclonal antibody, wherein said monoclonal antibody specifically binds an internal sequence in the N-terminal region of type 3 collagen;
determining the amount of binding of said antibody; and
correlating said amount of binding with values associated with normal healthy subjects, and/or with values associated with known responders to anti-TGFβI therapy, and/or with a predetermined cut-off value.

16. The method of claim 15, wherein the monoclonal antibody which specifically binds the an internal sequence in the N-terminal region of type 3 collagen specifically binds to the amino acid sequence PGIPGRNGDP (SEQ ID No: 2)

17. The method of claim 15, wherein the monoclonal antibody does not specifically recognise or bind to an elongated version of said C-terminal amino acid sequence which is PPGIPGRNGDP (SEQ ID No: 32) or PGIPGRNGDPG (SEQ ID No: 33), and/or a truncated version of said C-terminal amino acid sequence which is GIPGRNGDP (SEQ ID No: 34) or PGIPGRNGD (SEQ ID No: 35).

18. The method of claim 15, wherein the monoclonal antibody is raised against a synthetic peptide comprising or consisting of the amino acid sequence PGIPGRNGDP (SEQ ID No. 2).

19. The method of claim 1, wherein the patient sample is a biofluid.

20. The method of claim 19, wherein said biofluid is serum or plasma.

21. The method of claim 1, wherein the immunoassay is a competitive immunoassay.

22. The method of claim 21, wherein the competitive immunoassay is a radioimmunoassay, fluorescence immunoassay, or an enzyme-linked immunosorbent assay.

23. The method of claim 1, further comprising administering anti-TGFβI therapy to patient identified as likely to respond.

24. An agent which binds to the EMI domain of TGFβI or periostin or an analogue thereof.

25. The agent of claim 24, wherein the agent is an antibody.

26-28. (canceled)
